(19) United States
(12) Patent Application Publication
(10) Pub. No.: US 2024/0342253 A1
(43) Pub. Date: Oct. 17, 2024

(54) FIBRINOGEN COMPOSITIONS AND METHODS OF PREPARATION

(71) Applicant: Biotest AG, Dreieich (DE)

(72) Inventors: Oliver MANEG, Dreieich (DE); Florian SOCHOR, Dreieich (DE); Wolfgang MÖLLER, Dreieich (DE); Vera OTT, Dreieich (DE); Christoph SCHEICH, Dreieich (DE)

(21) Appl. No.: 18/681,561

(22) PCT Filed: Aug. 12, 2022
(86) PCT No.: PCT/EP2022/072679

§ 371 Date: Feb. 6, 2024

(30) Foreign Application Priority Data
Aug. 13, 2021 (EP) 21191286.0

(51) Int. Cl. A61K 38/36; A61K 9/00; A61K 9/16; A61K 9/19

(52) U.S. Cl. CPC A61K 38/363; A61K 9/0019; A61K 9/1611; A61K 9/1617; A61K 9/1623; A61K 9/19

(57) ABSTRACT

The present invention relates to the field of blood products, in particular, to fibrinogen and fibrinogen drug products. The invention provides a fibrinogen drug product in dry, e.g., lyophilized form having a residual moisture content of 2-5% (w/w). The inventors have found that said moisture content is advantageous for viral inactivation by dry heat, which leads to a particularly virus-safe and stable product. The invention further provides a fibrinogen drug product in dry, e.g., lyophilized form, that has an especially low number of subvisible particles (SVPs), and a batch of such drug products. It suitable for reconstitution of 1 g of fibrinogen in water for injection to obtain a fibrinogen solution comprising 6000 or less SVPs having a size of 10-100 μm and 600 or less SVPs having a size of 25-100 μm. Methods of preparing the drug products of the invention are also disclosed, as well as these drug products for use in treatment of fibrinogen-deficiency.

The present invention relates to the field of blood products, in particular, to fibrinogen and fibrinogen drug products. The invention provides a fibrinogen drug product in dry, e.g., lyophilized form having a residual moisture content of 2-5% (w/w). The inventors have found that said moisture content is advantageous for viral inactivation by dry heat, which leads to a stable product with high margins of safety towards infectious viruses, and a container comprising said drug product. The invention further provides a fibrinogen drug product in dry, e.g., lyophilized form that has an especially low number of sub-visible particles (SVP), and a container comprising said drug product. Also provided is a batch of such containers or drug products. Said drug product is suitable for reconstitution of one container comprising, e.g., 1 g of fibrinogen of said drug product in an aqueous solution, e.g., water for injection, to obtain a fibrinogen solution comprising not more than 6000 SVPs having a size of 10-100 μm and not more than 600 SVPs having a size of 25-100 μm. Methods of preparing the drug products of the invention are also disclosed, as well as these drug products for use in treatment of fibrinogen-deficiency.

Fibrinogen is the main structural protein in blood responsible for the formation of blood clots. During tissue and vascular injury, fibrinogen is converted enzymatically by thrombin to fibrin and then to a fibrin-based meshwork which forms the basis for the blood clot. Fibrin clots function primarily to occlude injured blood vessels to stop bleeding. Fibrin also binds and reduces the activity of thrombin. This activity provides a feed-back mechanism limiting excessive clotting. Fibrin also mediates blood platelet and endothelial cell spreading, tissue fibroblast proliferation, capillary tube formation, and angiogenesis and thereby promotes revascularization and wound healing. It can be used for treating haemostatic disorders.

The fibrinogen molecule circulates as a soluble plasma glycoprotein composed of two trimers, with each trimer composed of three different polypeptide chains, the fibrinogen alpha chain, the fibrinogen beta chain, and the fibrinogen gamma chain. Fibrinogen has a typical molecular weight of ˜340 kDa. The normal concentration of fibrinogen in human blood plasma is 150-400 mg/dl, with levels appreciably below or above this range associated with pathological bleeding and/or thrombosis (Wikipedia).

In case of fibrinogen-deficiency the blood's ability to form a clot is impaired, which leads to a greatly increased risk of critical bleeding and additionally to a delay in bleeding cessation.

In the case of severe congenital fibrinogen-deficiency, patients' ability to produce sufficient levels of functional fibrinogen is impaired or absent. These patients require frequent injections of fibrinogen concentrate. In acquired fibrinogen-deficiency, patients lose endogenous fibrinogen, which can lead to uncontrolled bleeding. A frequent cause is high blood loss during complex surgery, but also as a result of severe traumatic injuries. In this case fibrinogen must be administered intravenously to stop the bleeding by increasing fibrinogen above the critical level.

Thus, fibrinogen products and methods of preparing them are well-known in the art. For example, Haemocomplettan® is produced by CSL Behring (Marburg, Germany). WO 00/47621 A1 teaches a method for preparing a composition comprising fibrinogen and fibronectin. WO 2018/115800 A1, WO 2008/117746 A1, EP 0 085923 A1, EP 0 804 933 A2, WO 95/26749 A1, WO 98/55105 A1 and EP 0 345 246 A2 relate to fibrinogen compositions with different stabilizing agents.

WO 01/48016 A1, WO 2004/007533 A1, WO 2012/038410 A1, WO 2009/155626 A2 teach methods of producing fibrinogen compositions. For example, as fibrinogen is a rather sensitive protein with a tendency to form aggregates, WO 2006/015704 relates to thermal treatment of fibrinogen products, wherein a formation of aggregates is minimised.

In general, protein containing formulations may contain particulate matter, wherein the proteins of the formulation may self-aggregate to form particles (Carpenter et al., J Pharm Sci. 2009 April: 98 (4): 1201-1205). Particles may be visible or subvisible. Subvisible particles (SVPs) generally have a diameter of up to 100 μm. The upper size represents the limit of detection by the naked human eye. Therefore, the particles≤100 μm are called “subvisible”.

One aspect of product stability and quality thus is the presence of SVPs (Abraham et al., 2011. BioPharm International 24 (4)). Such particles, which may consist of aggregated proteins, and/or components shed from process materials or container closure systems, can directly impact the efficacy and immunogenicity of a drug product. Also, they often act as nucleation sites for further protein aggregation and/or lead to the development of larger particles by agglomeration. Measuring the size and concentration of SVPs within a formulation is an essential precursor to their effective control, and of growing importance as the industry works towards ‘zero defect’ and ‘essentially particle-free’ products (Carpenter et al., 2015, www.europeanpharmaceuticalreview.com/article/35952/meeting-biopharmaceutical-analytical-requirements-for-subvisible-particle-sizing-and-counting/). Current US Pharmacopoeia (USP) requirements for the light obscuration test<788>, the standard test for SVP analysis, specify that particulates≥10 μm in size are controlled at or below 6000 particles/container and particles≥25 μm are limited to at or below 600 particles/container. These limits are associated with concerns about particles blocking capillaries (average diameter around 7 μm) upon injection. Other health issues, e.g., increased immunogenicity, may further be present with SVPs of all sizes (Carpenter et al. 2009. Journal of Pharmaceutical Sciences 98 (4): 1201-1205).

At present, there are exceptions for plasma proteins, and for proteins for intramuscular and subcutaneous administration, which may comprise higher numbers of SVPs. However, it is clear that this is not due to lack of concerns, but rather due to the fact that it has so far not been possible to reproducibly and routinely produce plasma products comprising sufficiently low numbers of SVPs.

WO 2013/106772 A2 discloses methods for characterization of a population of particles using a particle analyzer and describes that generation of SVPs may be caused by the conditions of the preparation and/or packaging process.

WO 2016/057739 A1, WO 2014/100143 A2 and WO 2019/060062 A1 teach antibody compositions comprising low numbers of SVPs and fatty acid esters such as polysorbate and/or surfactants.

Still, there is a great need for plasma products, e.g., fibrinogen products, comprising low numbers of SVPs.

Fibrinogen is typically purified from plasma that cannot be guaranteed to be free of infectious viruses despite stringent donor screening and donation testing requirements, one other common problem is contamination with viruses. This necessitates virus removal or virus inactivation steps integrated into the manufacturing process. Removal can e.g., be performed by nanofiltration (e.g., in the case of Fibclot® from LFB SA, Courtaboeuf Cedex, France), which has the disadvantage that the filters are expensive and often get blocked. Chromatography can also be used. Virus inactivation can be based on solvent/detergent (S/D) treatment, pasteurization or heat treatment (e.g., in WO 97/42980 A1), inactivation with an acidic pH or with irradiation, e.g., with UV light. Heat inactivation in the presence of saccharose, as performed for the CSL Behring product Haemocomplettan® may be considered to be undesirable due to the issues arising for diabetic patients. Combinations of effective methods for virus inactivation/removal are also often used in the art as they are mandatory before the European Medicines Agency (EMA) against lipid-enveloped viruses.

Virus in Factor VIII products is, e.g., inactivated with heat treatment of a lyophilised composition in EP 0 844 005 A1 at a residual moisture content of 0.8%. JPS6289628 A describes drying fibrinogen until a residual moisture content of 0.05-3% and a heat treatment at 60° C. for 65-90 h in the presence of a disaccharide. WO 93/05067 A1 teaches polysorbate 80 as an antiviral substance and solubilisator in a topical fibrinogen complex. Success of virus inactivation depends on several factors in the composition and preparation of a drug product, and needs to be experimentally verified, e.g., by virus spiking experiments.

In light of the state of the art, the inventors addressed the problem of providing advantageous fibrinogen drug products that address one or more of these problems. In particular, the inventors addressed the problem to provide a particularly safe product for e.g. intravenous administration, which is an active, long-time stable and virus-safe fibrinogen product. Preferably, the fibrinogen product should be characterized by a good solubility, a very low content of SVPs, and it should be substantially free of secondary proteins for stabilization or the like. Moreover, the blood product should be producible in a well standardized process in a well reproducible way.

### Fibrinogen Drug Products of the Invention

This problem is solved by the subject matter of the invention, e.g., the subject-matter of the claims. Advantageously, the invention provides fibrinogen drug products that are particularly safe to use with regard to virus-safety and/or the number of SVPs, or both, and containers comprising such drug products.

In one embodiment, the invention provides a container comprising a fibrinogen drug product in dry, preferably, lyophilized form having a residual moisture content of 2-5% (w/w), preferably 2.5-4% (w/w). A residual moisture content of 2.5-3.5%, e.g., about 3% (w/w) has been shown to be particularly favourable with regard to optimization of both virus inactivation and stability. Thus, more preferably, the fibrinogen drug product of the invention is dry, preferably, is lyophilized, and has a residual moisture content of 2.5-3.5% (w/w), preferably about 3% (w/w). The moisture content is preferably determined according to the method of NIR spectroscopy. Alternatively, it can be determined with the method of Karl Fischer. If not mentioned otherwise, % residual moisture content relates to w/w (weight H₂O/weight of drug product).

As known to the skilled person, a drug product is a pharmaceutical composition, namely, a finished dosage form which is ready for administration, if necessary, after further reconstitution in a solvent, to the patient as a pharmaceutical, and/or ready for sale and/or distribution to the patient or a medical practitioner. Generally, a drug product is prepared from a bulk drug substance. In the present disclosure, the drug product of the invention, or a container comprising the same is also referred to as the product of the invention.

According to the invention the drug product is dry, which is not intended to mean absolute absence of water, but, rather qualifies that the product is in solid form, and typically, has been dried. Compared to other fibrinogen drug products from the state of the art, it has a relatively high moisture content of 2-5% (w/w), as specified above. Lyophilisation is a preferred option of drying, however, the product can alternatively be spray-dried or spray-freeze-dried. Moisture refers to water. The drug product does not comprise any other solvents in significant amount, in particular, no solvents (or other ingredients) in an amount incompatible with pharmaceutical use, e.g., intravenous administration.

The inventors have surprisingly found that the efficacy of virus inactivation by dry heat, in particular, the efficacy of virus inactivation for non-enveloped viruses, is significantly increased with the specified moisture content and that the drug product of the specified moisture content produced by said virus still has a high stability and good solubility. The particularly efficient virus-inactivation and high stability and solubility are demonstrated in the examples and figures below. Thus, the drug product of the invention is particularly virus-safe, is highly stable and has a good solubility in reconstitution.

As used herein the term “fibrinogen” refers to the main structural protein responsible for the formation of clots as present in blood plasma and preferably refers to the whole glycoprotein form of fibrinogen. Preferably, it refers to plasmatic fibrinogen, i.e. a fibrinogen, which is derived from plasma. More preferably, fibrinogen is a human plasmatic fibrinogen. Alternatively, the fibrinogen used in the present invention can be recombinantly produced.

Preferably, the drug product is packaged in dosage unit form. Typically, for fibrinogen, this means that about 1 g of fibrinogen is packaged. Before administration to a patient, the dry, e.g. lyophilized, fibrinogen drug product is typically reconstituted in a solvent, in particular, an aqueous solvent to obtain a solution. 1 g of fibrinogen may be reconstituted e.g., in water for injection, a buffer or plasma, typically, in 50 mL thereof to provide a solution of total protein, mainly, fibrinogen at about 20 g/L.

In another embodiment, the invention provides a container comprising a fibrinogen drug product in dry form, wherein the fibrinogen drug product is suitable for reconstitution in an aqueous solution, preferably water for injection, and wherein a fibrinogen solution resulting from said reconstitution of 1 g of fibrinogen of said drug product comprises not more than 6000 SVPs having a size of 10-100 μm and not more than 600 SVPs having a size of 25-100 μm. “suitable for reconstitution” means that, upon reconstitution of the content of one container in the solvent, especially in water for injection, a fibrinogen solution comprising not more than 6000 or less SVPs having a size of 10-100 μm and not more than 600 SVPs having a size of 25-100 μm is obtained. Here, the amount of SVPs refers to the absolute amount of SVPs within the container, respectively within the respective dosage unit (e.g., 50 mL, or 50-100 mL).

Typically, the fibrinogen drug product comprises about 50-98% (w/w) of active fibrinogen, preferably, 70-95%, e.g., 80-90%. Thus, often, if it is desired to package 1 g of (active) fibrinogen, more than 1 g of drug product is comprised in the container, e.g., about 1.5-2 g of fibrinogen drug product. The drug product typically further comprises excipients, e.g., as described below. Preferably, the recited maximum concentrations of SVPs applies upon reconstitution of one container of fibrinogen drug product comprising, e.g., 1 g of fibrinogen. Said maximum concentrations of SVPs may also apply upon reconstitution of one container of fibrinogen drug product comprising, e.g., 2 g of fibrinogen or more, optionally, 3 g or 5 g of fibrinogen.

In one embodiment, the invention provides a fibrinogen drug product in dry, preferably, lyophilized form having a residual moisture content of 2-5% (w/w), optionally, a residual moisture content of 2.5-3.5% (w/w), e.g., about 3% (w/w), wherein the drug product is suitable for reconstitution in an aqueous solution, preferably water for injection, and wherein a fibrinogen solution resulting from said reconstitution of 1 g of fibrinogen of said drug product comprises not more than 6000 SVPs having a size of 10-100 μm and not more than 600 SVPs having a size of 25-100 μm.

The official requirements for SVPs according to Ph. Eur. 2.9.19 and USP 788 (which provide standards for particulate matter in injections) are, for the first time, met for a fibrinogen drug product with the product of the invention. Ph. Eur. 2.9.19 and USP 788 generally require≤6000 particles per 50 mL container for particles≥10 μm and ≤600 particles per 50 mL container for particles≥25 μm, which corresponds to 120 particles≥10 μm per mL and 12 particles≥25 μm per mL.

The number of fibrinogen molecules (fibrinogen concentration in the reconstituted drug product (DP): 20 g/L, molecular weight about 340 kDa, concentration about 60 μmol/L) with a length of 0.05 μm (Hall et al, 1959) may, e.g., be about 17.7*10¹⁸ per mL.

Reconstitution is carried out with a pharmacologically acceptable solvent, typically, an aqueous solvent. Preferably, water for injection is used, but reconstitution can also be, e.g., in physiological saline or in a buffer such as PBS. During reconstitution, the fibrinogen drug product is dissolved in the solvent. Reconstitution can e.g. be carried out by adding the solvent into the container, optionally, including mixing, e.g., by with a vortex mixer, by shaking, or by drawing up into a syringe, optionally, repeatedly. Reconstitution for the purposes of analysis of the number of SVPs does not comprise filtration.

In accordance with Ph. Eur. 2.9.19 and USP 788, the amount of SVPs is measured with light obscuration, e.g., by the particle counter Hiac Model 9703+ (Beckman, Krefeld, Germany) equipped with a Hiac Model HRLD-400 sensor 2 to 400 μm size range. For each measurement, 5 mL of the protein solution are sucked through a capillary tube and passed by the sensor. The first measurement is used to flush the system and the results are discarded. The mean of the following four measurements is used to determine the number of SVPs in the solution. It is important to note that the protein solution is not filtered before the measurements.

For the final fibrinogen drug product (DP), the numbers of SVPs≥10 μm and ≥25 μm are preferably determined. For this, the freeze-dried DP is preferably reconstituted with 50 ml water for injection (WFI). The reconstituted solution is transferred to the particle counter without any further filtration. Every sample is preferably measured three times. The mean value of the total particle amount per container is reported. As the SVPs≥25 μm are also included in the group of SVPs≥10 μm, the number of SVPs≥10 μm is always higher than the number of SVPs≥25 μm.

Due to the low number of SVPs contained in the drug product of the invention, it is possible to administer said drug product safely without previous filtration. This is particularly advantageous when time is of the essence. Filtration may however still be carried out, e.g., for solution of the dry product in a manner that avoids formation of foam.

The present invention for the first time provides a reproducible method of producing such fibrinogen drug products and containers comprising them. Thus, the invention also provides a batch of containers comprising a fibrinogen drug product in dry form, wherein, for at least 10 containers from said batch, the fibrinogen drug product is suitable for reconstitution in an aqueous solution, preferably water for injection, and wherein a fibrinogen solution resulting from said reconstitution of 1 g of fibrinogen of said drug product comprises not more than 6000 SVPs having a size of 10-100 μm and not more than 600 SVPs having a size of 25-100 μm. Preferably, these conditions apply for at least 100 containers from the batch of the invention, optionally, for at least 200 containers. The limits may also be met for at least 500 containers from the batch of the invention, optionally, for substantially all containers of said batch. The containers may be randomly selected from the batch, preferably, equally, or approximately equally from the half of the batch first filled into containers and the half of the batch then filled into the containers.

The invention also provides a batch of containers comprising a fibrinogen drug product in dry form, wherein, for at least the 10 final % of containers from the batch, preferably, the at least the final 20% of containers from said batch, optionally, substantially all containers of said batch, the fibrinogen drug product is suitable for reconstitution in an aqueous solution, preferably water for injection, and wherein a fibrinogen solution resulting from said reconstitution of 1 g of fibrinogen of said drug product comprises not more than 6000 SVPs having a size of 10-100 μm and not more than 600 SVPs having a size of 25-100 μm. Final in this context means filled last.

Preferably, the fibrinogen drug product used in the invention contains even less SVPs. For example, it may be suitable for reconstitution in an aqueous solution, preferably water for injection, and wherein a fibrinogen solution resulting from said reconstitution of 1 g of fibrinogen of said drug product comprises not more than 3500 SVPs having a size of 10-100 μm and not more than 60 SVPs having a size of 25-100 μm. Optionally, the fibrinogen drug product used in the invention is suitable for reconstitution in an aqueous solution, preferably water for injection, and wherein a fibrinogen solution resulting from said reconstitution of 1 g of fibrinogen of said drug product comprises not more than 3500 SVPs having a size of 10-100 μm and not more than 35 SVPs having a size of 25-100 μm. The inventors could show that the invention also provides a fibrinogen drug product of the invention that is suitable for reconstitution in an aqueous solution, preferably water for injection, and wherein a fibrinogen solution resulting from said reconstitution of 1 g of fibrinogen of said drug product comprises not more than 2500 SVPs having a size of 10-100 μm and not more than 30 SVPs having a size of 25-100 μm.

Insofar as the invention relates to a batch of containers comprising fibrinogen drug product of the invention, said fibrinogen drug product has a residual moisture content of 2-5% (w/w), preferably, 2.5-3.5% (w/w). This means that each drug product in the batch has said residual moisture.

The fibrinogen drug product of the invention is packaged in a container such as a vial, e.g., a glass vial or a plastic (e.g., polyethylen) vial. Preferably, the container is a glass container. The container may, e.g., have a volume that allows for addition of at least 50 mL of a solution. Preferably, the container has a nominal filling volume of 100 mL. The container may also be a syringe, e.g., a single chamber syringe. It may also be a double chamber syringe comprising the fibrinogen drug product in one of the chambers.

The invention provides for a kit comprising a drug product of the invention in a suitable container, e.g., a vial, and/or a suitable transfer device. More preferably, said transfer device is a device facilitating clean or sterile transfer of a suitable solvent (e.g. water for injection) from a container comprising said solvent into the container comprising the drug product. Even more preferably, said transfer device also facilitates transfer of the dissolved product to a device for administration, e.g. a syringe. Such transfer devices are commercially available (e.g., Mix2Vial® (West), Nextaro® (SFM)) and known to the skilled person. Said transfer devices optionally comprises one or more filters, e.g. filters capable of removing bacteria and/or depleting any particles in the solvent and/or the product. Such filter(s) may e.g. be used to ensure removal of any particles having entered the drug product or the solvent as a result of penetrating the closure of the container (e.g. material from a rubber stopper resulting from penetration with a needle).

Mesh filter or a membrane filters may be used as deemed appropriate. Pore size of such filter(s) may be in the range of 3 μm to 25 μm, preferably in the range 4 to 10 μm. The term “pore size” relates to the nominal pore size.

For filter(s) through which the dissolved drug product is passed, preferably a membrane filter is used. Preferably, said filter has a pore size of 3 to 10 μm, more preferably 4 to 9 μm, e.g. 5 or 8 μm. The inventors have found that particularly good results can be achieved with membrane filters made from acrylic copolymer.

Thus, the present invention also relates to a kit comprising a drug product of the invention in a suitable container (e.g., a vial), and a suitable transfer device as described above. Preferably, said kit also comprises a suitable container comprising an aqueous solvent (e.g. water for injection) for dissolving the drug product. For example, the present invention also relates to a kit comprising (i) a drug product of the invention in a suitable container (e.g., a vial), (ii) a suitable transfer device comprising a filter for filtering the drug product having pore size of 3 to 10 μm, preferably wherein the membrane is an acrylic copolymer membrane, and (iii) a suitable container comprising an aqueous solvent (e.g. water for injection)

The container of the invention typically contains fibrinogen drug product containing 1 g of fibrinogen, but it may also comprise other amounts, e.g., 0.5-10 g or 1-5 g, such as 2 g or 5 g of fibrinogen.

Preferably, the container, e.g., the vial, is suitable for reconstitution at a concentration of 20 g fibrinogen/L in 50 mL water for injection. The reconstitution to measure the number of SVPs defined herein is typically also carried out at said concentration.

The fibrinogen drug product of the invention is dry, preferably, lyophilized. It may also be spray-dried or spray-freeze-dried.

The fibrinogen drug product used in the invention may comprise, in addition to fibrinogen and the residual moisture specified,
a) polysorbate,
b) optionally, a bulking agent,
c) optionally, an amino acid and/or
d) optionally, a salt.

For example, the fibrinogen drug product used in the invention preferably comprises polysorbate, e.g, polysorbate 80, as the inventors could show that the polysorbate contributes to reduction of virus load upon dry heat treatment at the preferred residual moisture content, and may further help to reduce SVPs. Polysorbate 20 is an alternative.

The fibrinogen drug product or batch of fibrinogen drug products of the invention may also comprise a bulking agent, optionally, trehalose. Preferably, the bulking agent also acts as a lyoprotectant. Mannose can alternatively be used, but the inventors have found that trehalose leads to a better cake and better solubilisation characteristics.

The fibrinogen drug product or batch of fibrinogen drug products of the invention may further comprise an amino acid, optionally, arginine. The amino acid preferably has a stabilizing effect. Lysine can be alternatively used, but the inventors have found that arginine leads to a better cake and better solubilisation characteristics.

Further, the fibrinogen drug product or batch of fibrinogen drug products of the invention may comprise a salt, preferably sodium chloride, sodium citrate, or a combination thereof, optionally, sodium chloride and sodium citrate. The salt may, e.g., serve as a buffer and/or to provide an isotonic solution. Preferably, the composition does not comprise significant amounts of potassium due to its toxicity.

Thus, the fibrinogen drug product used in the invention may, e.g., comprise
a) polysorbate 80,
b) trehalose,
c) arginine and
d) a salt preferably sodium chloride, sodium citrate and a combination thereof.

Preferably, the fibrinogen drug product used in the invention comprises polysorbate 80, trehalose, arginine, sodium, chloride, citrate and residual moisture. It may further comprise calcium, preferably, at a concentration of less than 1 mM. A higher concentration of calcium may contribute to undesired complex formation. The fibrinogen drug product or batch of fibrinogen drug products of the invention may further comprise additional plasma proteins selected from the group comprising albumin, fibronectin, a2-macroglobulin, immunoglobulins such as IgG, IgA or IgM, Von Willebrand factor (vWF), fibrinopeptide A and D-Dimer, e.g., traces thereof, preferably in the approximate concentrations provided in the Examples below.

A preferred fibrinogen drug product used in the invention substantially consists of the recited components, i.e., fibrinogen, a polysorbate such as polysorbate 80, a bulking agent such as trehalose, an amino acid such as arginine, a salt such as sodium chloride and/or sodium citrate, or plasma protein(s) selected from the group comprising albumin, fibronectin, a2-macroglobulin, immunoglobulins such as IgG, IgA or IgM, Von Willebrand factor, fibrinopeptide A and D-Dimer, and residual moisture.

For example, in a preferred embodiment, a fibrinogen drug product used in the invention may comprise, upon reconstitution of 1 g of fibrinogen of said drug product at 20 g/L in water for injection, as described herein, 95-135 mmol/L Na, less than 1 mmol/L Ca, 100-160 mmol CI, 3-7 mmol/L citrate, 25-55 mmol/L arginine, 22-38 mmol/L trehalose and 0.03-0.07% polysorbate 80.

In particular, preferably, the drug product of the invention does not comprise significantly more albumin than recited, e.g., it preferably comprises, upon reconstitution at 20 g/L fibrinogen, less than 0.5 g/L albumin, preferably, less than 0.15 g/L albumin. Thus, it is possible to provide suitable doses of fibrinogen and albumin to a patient separately, and, further, it is not required to perform a quality control process for both fibrinogen and albumin. Preferably, none of the non-fibrinogen plasma proteins recited above is contained in a concentration higher than 0.5 g/L upon reconstitution at 20 g/L fibrinogen to allow for separate dosing.

It is additionally preferred that the fibrinogen drug product or batch of fibrinogen drug products of the invention does not comprise saccharose and/or glutamate, preferably, neither saccharose nor glutamate. Saccharose is problematic, in particular, in the context of diabetes. Concerns about glutamate are discussed in the context of the “Chinese restaurant syndrome”.

The inventors have found that the fibrinogen drug product used in the invention is very stable. The inventors have found that advantageously, the drug product of the invention is stable at 2-25° C. for at least 6 months, preferably, for at least 1 year, optionally, for at least 5 years, when stored in dry form. Moreover, as shown in the examples below, after reconstitution, it can be stored at 2-8° C. for at least 8 weeks without significant degradation. Stable preferably means that the drug product maintains at least 80%, optionally at least 90% of specific fibrinogen activity originally contained.

The fibrinogen drug product or batch of fibrinogen drug products of the invention preferably comprises less than 20% of aggregates, preferably, less than 10% of aggregates. Aggregates are typically, multimers of fibrinogen, e.g., dimers, trimers etc. The proportion of aggregates can be determined by HP-SEC analysis. The proportion is specified as the detected amount of fibrinogen present in the form of aggregates compared to the total amount of fibrinogen.

In one embodiment, the invention also provides a fibrinogen solution of the invention obtainable by reconstituting the dry drug product of the invention in an aqueous solvent, especially in water for injection, wherein the solution comprises the low number of SVPs as defined herein. Also provided is a method of reconstituting said fibrinogen solution comprising adding water for injection or another aqueous solvent to the dry drug product of the invention.

### Method of Preparing Containers Comprising the Fibrinogen Drug Product of the Invention

In another embodiment, the invention provides a method for producing a container comprising the fibrinogen drug product of the invention, comprising steps of
a) sterile filtering a bulk solution of fibrinogen drug substance,
b) receiving the sterile filtered bulk solution in a receiving tank comprising, optionally, a stirrer having a stirring means,
c) optionally, stirring the bulk solution in the receiving tank when the stirring means is submerged in said bulk solution,
d) filling containers with a pre-determined amount of the solution, e.g., using a pump,
e) lyophilizing the solution in the containers to obtain a lyophilized drug product, and
f) treating the lyophilized drug product with dry heat, and
g) optionally, packaging the containers comprising the drug product.

In one embodiment, the method comprises
a) sterile filtering a bulk solution of fibrinogen drug substance,
b) receiving the sterile filtered bulk solution in a receiving tank,
c) no stirring of the bulk solution in the receiving tank,
d) filling containers with a pre-determined amount of the solution, e.g., using a pump,
e) lyophilizing the solution in the containers to obtain a lyophilized drug product, and
f) treating the lyophilized drug product with dry heat, and
g) optionally, packaging the containers comprising the drug product.

The inventors have surprisingly found that stirring of the bulk solution before and/or during the filling step is not necessarily required. The solution stays homogenous during the whole filling process, e.g., over 2-3 hours without stirring. Samples taken during filling show a constant concentration of fibrinogen and other ingredients over the whole filling process even in the absence of stirring. The inventors have also found that reduction or avoidance of the stirring process reduces the amount of SVPs.

Still to avoid any risk of inhomogeneity of the product, stirring for a short time and/or under mild conditions can also be included in the method of the invention. Thus, typically, the method comprises steps of
a) sterile filtering a bulk solution of fibrinogen drug substance,
b) receiving the sterile filtered bulk solution in a receiving tank comprising a stirrer having a stirring means,
c) stirring the bulk solution in the receiving tank when the stirring means is submerged in said bulk solution,
d) filling containers with a pre-determined amount of the solution, e.g., using a pump,
e) lyophilizing the solution in the containers to obtain a lyophilized drug product, and
f) treating the lyophilized drug product with dry heat, and
g) optionally, packaging the containers comprising the drug product.

Stirring has the advantage that inhomogeneity of the solution, and consequently, inhomogeneous packaging, is very reliably avoided. However, the inventors found that even without stirring the solution was sufficiently homogenous.

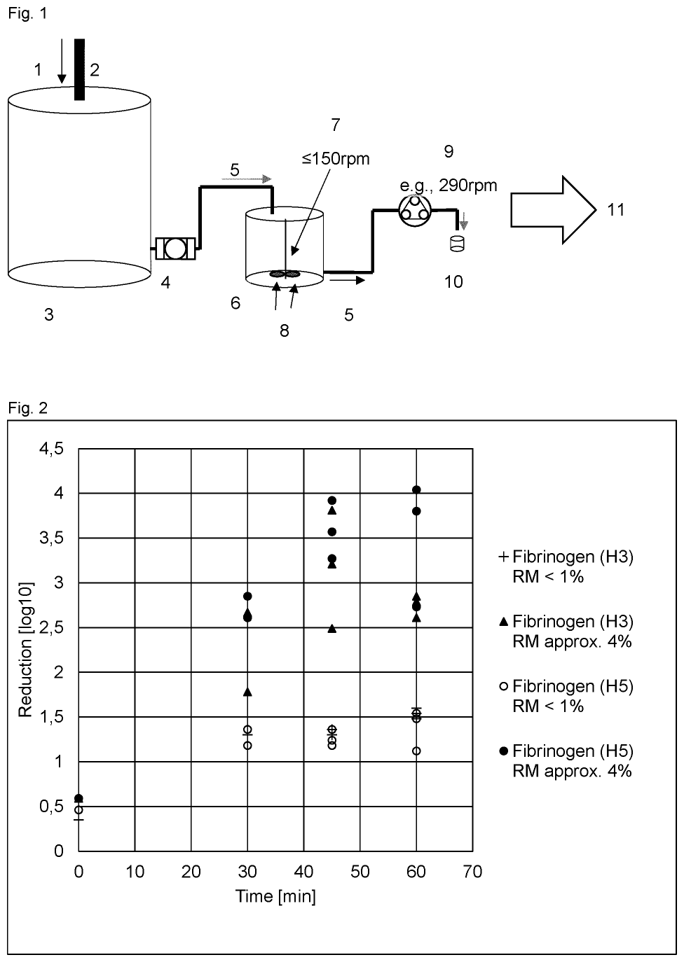

An exemplary filling line that may be used in the method of the invention is shown in FIG. 1.

The sterile filtration in step a) is preferably carried out with a filter having a mesh size of 0.2 μm, thus, the solution is sterile filtered. The solution of fibrinogen drug substance is typically passed through said filter from a feeding tank, which may be a stainless steel tank or plastic container (such as a bag), e.g., capable of holding 80 L. The solution is pressed through the filter by a pressure line. The transfer pressure is at most 600 mbar (600 hPa). Optionally, after sterile filtration, the solution is directly passed into the receiving tank (step b).

In step b), the sterile filtered bulk solution is received in a receiving tank, e.g, a stainless steel tank. The volume may be, e.g., 50 L. The receiving tank optionally comprising a stirrer having a stirring means, preferably, a stirring bar or rod. In the context of the invention, the term “a” is understood to refer to “at least one”. Thus, the stirring tank may also comprise two or more stirrers. The stirrer may also have at least one stirring means, e.g., one stirring blade. Preferably, it has two or more stirring means, e.g., stirring blades. In a two-bladed stirrer, the blades are typically opposite each other. The stirring means, e.g., stirring blades preferably mix the solution by rotating in a horizontal manner. The stirring blades may be affixed to a central drive shaft that typically rotates, and thus moves the stirring blades. The stirring means may also be a stir bar, e.g, a magnetic stir bar. The stirring means may also be a swinging plate, or a plurality of swinging plates.

In optional step c), the bulk solution is stirred in the receiving tank when the stirring means is submerged in said bulk solution. Thus, the solution is not stirred before the stirring blade or other stirring means (all stirring means or blades, if there are several) are submerged. Consequently, in contrast to prior art methods, the solution is not stirred before sufficient solution has been received in the receiving tank that the stirring means is submerged. This has the effect that the surface of said bulk solution is not broken by the stirring means upon stirring. Thus, foam formation is prevented, and the shear force to which the solution is subjected is minimized. If the solution is stirred during filling, when the solution from the receiving tank is filled into the containers and the level of solution in the receiving tank falls again, the stirring is also stopped before the level of the solution falls below the stirring means, so that that the surface of said bulk solution is not broken by the stirring means upon stirring.

The time of stirring is preferably limited to at most 1 hour, more preferably, to at most 10 min, most preferably, at most 5 min for a 50 L receiving tank to further minimize the exposure to shear stress. The time can be adapted to the volume of the solution and the tank, e.g., for a larger tank and/or a larger amount of solution, the time of stirring can be longer.

Small scale experiments have shown that the rate or velocity of stirring, the time of stirring and the geometrical characteristics of the stirrer can influence the number of SVPs formed.

Preferably, the stirrer, e.g., a stirrer with two horizontal stirring blades fixed to a rotating vertical driving shaft, as described above, is turned at a speed of at most 150 rpm, e.g., 30-100 rpm or 50-80 rpm. Preferably, the shear force to which the solution is exposed is not higher than the shear force to which the solution in a cylindrical stainless steel receiving tank of 50 L is exposed with a preferred stirrer having two stirring blades as defined above turned at a rate of at most 150 rpm, e.g., 80 rpm.

In a particularly preferred embodiment, in a cylindrical stainless steel receiving tank of 50 L, e.g., with a filling line KS 1025, e.g., model AS18.3 from Bausch & Ströbel, Ilshofen, Germany, a rate of stirring of ≤150 rpm, e.g, 80 rpm, for 0-5 min was shown to minimize formation of SVPs.

It was further shown that no stirring during filling is required. If additional bulk drug solution is added to the receiving tank, it is possible to restart stirring for a limited time, as described above. In this case, the maximal stirring times apply for the total stirring to ensure that none of the drug solution is stirred for too long. If additional solution has to be added, it is advantageous to do so while there is still so much solution in the receiving tank that the stirring blades, if present, do not break the surface of the solution if turned.

The inventors have surprisingly found that this smooth or gentle stirring, and thus, a minimization of the shear force to which the fibrinogen solution is exposed leads to the low number of SVPs that is advantageously comprised in the drug product used in the invention. Tests carried out by the inventors have shown that the shear forces to which the fibrinogen solution is exposed after the sterile filtration may lead to a significant increase in the number of SVPs in the reconstituted product. In particular, the stirring in the receiving tank is a critical step. While stirring may be important to obtain a homogenous product, too much stirring, or stirring under the wrong conditions, e.g., when the surface of the solution is broken or foam generated, was shown to lead to a higher number of SVPs.

In step d), containers are filed with a pre-determined amount of the solution using a pump or by other means. The pump may be a peristaltic pump used at a rate of up to 300 rpm, e.g., 200-290 rpm, e.g., at most 270 rpm. The pre-determined amount of fibrinogen in the container preferably is 1-5 g, optionally, 1 g, 2 g or 3 g, e.g., 1 g.

In step e) the drug substance in the filled containers, e.g., vials is lyophilized. Preferably, the containers are loaded into a pre-chilled freeze-dryer. This allows for a quick freezing of the protein solution and results in a favorable, porous cake structure, which improves water sublimation. In the method of the invention, the lyophilisation conditions are preferably chosen to obtain a residual moisture content after lyophilisation of 2-5% (w/w), optionally, 2.5-4%, or 2.5-3.5% (w/w), e.g., about 3% (w/w).

The residual moisture content of the lyophilised drug product used in the invention mainly depends on the conditions of lyophilisation. The ingredients of the formulation, e.g., polysorbate 80 only have an influence on the residual moisture content as far as the conditions of lyophilisation may need to be adapted to obtain the desired residual moisture content.

The particular advantage of the relatively high residual moisture content of the preferred drug product of the invention is that the inventors found that due to the high residual moisture content virus inactivation is particularly effective during a dry heat treatment carried out after lyophilisation. Normally, a high residual moisture content is detrimental for product integrity, however, the inventors surprisingly found that the product of the invention having the defined residual moisture content is highly stable. A product having a residual moisture content of 2.5-3.5% has been shown to be optimal with regard to both parameters.

The improved virus inactivation by dry heat treatment could be shown for non-enveloped viruses with the example of Porcine Parvovirus (PPV). Studies with enveloped viruses such as HIV (Human Immunodeficiency Virus) or BVDV (Bovine Virus-diarrhoe Virus) do not show this effect so clearly. This may be due to the other virus-inactivating effects, e.g., polysorbate that is preferably contained in the product of the invention, that already lead to a very good virus inactivation of enveloped viruses upon lyophilisation, i.e., before dry heat treatment, with all analysed residual moisture contents, i.e., even if the residual moisture content is lower. Thus, in the preparation of fibrinogen drug products, non-enveloped viruses are preferably inactivated by dry heat treatment of a fibrinogen solution lyophilised to contain 2-5% residual moisture content, optionally, in the presence of polysorbate, e.g., polysorbate 80. Enveloped viruses are preferably inactivated by lyophilisation of a fibrinogen solution in the presence of polysorbate, e.g., polysorbate 80, and/or by UV-C treatment, as described herein. Preferably, all of these measures are carried out and contribute to provision of a virus-safe product.

In a preferred embodiment, the lyophilisation method of the invention comprises steps of
a) freezing (preferably, quick freezing) at −29° C. or less, for at least 4 hours, preferably, −50° C. or less for about 8 hours,
b) primary stepwise drying at −10° C. or less and at least 40 μbar (4 Pa), wherein preferably, temperatures increase in each step, starting with less than −25° C. and at least 200 μbar (20 Pa), preferably, 280 μbar (29 Pa),
c) secondary drying at 17 to 23° C. for at least 2 hours.

As specified above, preferably, the residual moisture content after said lyophilisation is 2-5% (w/w).

The lyophilisation method may more specifically comprise steps of
a) freezing at −52° C. or less, for 8 h or more,
b) primary stepwise drying, the first step being carried out at about −36° C. at about 280 μbar (28 Pa) for about 5-48 h, the second step being carried out at about-23° C. at about 70 μbar for about 25-45 h, and the third step being carried out at about −10° C. at about 40 μbar (4 Pa) for about 45-84 h, e.g., the first step being carried out at about −36° C. at about 280 μbar (28 Pa) for about 45 h, the second step being carried out at about −23° C. at about 70 μbar for about 45 h, and the third step being carried out at about −10° C. at about 40 μbar (4 Pa) for about 45 h,
c) secondary drying at about 20° C. at about 10 μbar (1 Pa) for about 3-4 h.

Alternatively, primary stepwise drying in step b) can be carried out, e.g., with a first step being carried out at about −36° C. for about 5 h and about 280 μbar, the second step being carried out at about −23° C. at about 70 μbar for about 25 h, and the third step being carried out at −10° C. at about 40 μbar for about 78 h.

In the method of the invention, in step f), a dry heat treatment is carried out. The treatment with dry heat may e.g., comprise heating the drug product to 100° C.+/−1.5° C., e.g., 99-100° C. The treatment with dry heat may be performed for 20-60 min, preferably, for 20-40 min. Good results have been obtained with 30+/−3 min. The treatment with dry heat is preferably performed in a steam autoclave, of course, after closing the container, e.g., with a rubber stopper, which may be pierceable.

The drug product is optionally packaged in step g). A package insert may be added, e.g., specifying that the drug product is to be dissolved in water for injection at 20 g fibrinogen/L, and/or that it is for intravenous administration. A filter may be packaged together with the container of drug product.

A preferred method for preparing the container comprising the drug product of the invention may comprise the following steps:

After sterile filtration (0.2 μm) of the drug substance, i.e., the formulated product, it is filled into containers, e.g., vials. For filling, preferably, the drug substance is received in a receiving tank and, optionally, carefully stirred while avoiding formation of foam while the stirring blades or other stirring means of the stirrer are covered with drug substance solution, e.g., for at most 5 min, or not stirred at all. The stirrer is run at ≤150 rpm, preferably ≤80 rpm. The drug substance is then filled into the containers, preferably, with a peristaltic pump at ≤290 rpm, e.g., ≤270 rpm. Typically, 32 ml are filled into the containers, e.g., corresponding to about 1 g of fibrinogen. This is followed by a lyophilisation to a residual moisture content of 2-5%, and then a dry heat treatment at about 30 min, 99-100° C. is performed for further virus inactivation. The container comprising the drug product may then be packaged.

Optionally, the method of the invention further comprises steps of preparing fibrinogen drug substance, before step a). For example, the fibrinogen may be prepared according to the method described below, or in the examples. It may also be prepared according to other methods, e.g., a method known in the art.

The starting material for manufacturing fibrinogen typically is human plasma. A cryoprecipitate of human plasma may be obtained by thawing frozen plasma at 0-4° C. and separation of the precipitate by centrifugation or other separators.

Per kg of cryoprecipitate a mixture of 2.91 kg of water (WFI), 114 g ethanol 25% (v/v) and 9,000 IU heparin may be prepared. The cryoprecipitate may be added to the WFI/ethanol/heparin solution under stirring. The pH value may be adjusted to 7.0.

108 g of a 2% aluminium hydroxide suspension may be added per kg of cryoprecipitate used, and the mixture stirred at 22.5° C. The pH value may be adjusted to 6.55 and subsequently centrifuged by continuously operating centrifuges.

1% polysorbate 80 and 0.3% Tri-n-butyl phosphate may be added while stirring. The protein solution may be stirred at 25.0° C. over a period of at least 8 hours.

The anion-exchange gel Toyopearl TSK DEAE-650 (hydroxylated methacrylic polymer beads as matrix material with diethylaminoethyl groups) may be used for further purification by column chromatography. The protein loading may be about 50±10 mg of protein/mL anion exchange gel.

The chloride content of the protein solution may be adjusted to 120 mmol/L by addition of NaCl solution. The protein solution may be applied to the column. The flow through fraction contains fibrinogen, which may be collected for further processing.

The resulting fibrinogen solution (flow through) may be subjected to glycine precipitation. To precipitate fibrinogen, glycine may be added to a final concentration of 1.2 M. NaCl may be added to a final concentration of 2 M. The fibrinogen containing precipitate may then be separated by centrifugation. The fibrinogen paste may be stored at a temperature≤−70° C.

The precipitate may be resuspended in a buffer (15 mM tri-sodium citrate dihydrate, pH value: 6.9+/−0.1, conductivity: 3.3+/−0.5 mS/cm). The composition beside the other proteins (e.g. 0.7-0.9 U/mg vWF) comprises TnBP, polysorbate 80, glycine and NaCl. The composition may be filtered and subjected to an UV-C treatment for virus inactivation using devices such as the UVivatec device (Sartorius Stedim Biotech). The irradiation is preferably performed at 254 nm+1 nm using 125-200 J/m².

For a following cation exchange chromatography step the column (POROS™ 50 HS) may be equilibrated with equilibration buffer (15 mM tri-sodium citrate dihydrate, 65 mM sodium chloride, pH value: 6.5+/−0.1, conductivity: 9.0+/−1.5 mS/cm, 2-5 column volumes).

The liquid phase containing fibrinogen resulting from the UV irradiation step may be prepared by adjusting the composition to 15 mM tri-sodium citrate, pH value: 6.5+/−0.1 and conductivity: 9.0+/−1.5 mS/cm. The column may be loaded with 10-20 g/l protein per liter gel volume.

The column may be rinsed with wash buffer (15 mM tri-sodium citrate dehydrate, 65 mM sodium chloride, pH value: 6.5+/−0.1, conductivity: 9.0+/−1.0 mS/cm, 2-5 column volumes).

Then the fibrinogen may be eluted using elution buffer (7.5 mM tri-sodium citrate dihydrate, 150 mM sodium chloride, 75 mM L-arginine monohydrochloride, pH value: 7.0+/−0.1, conductivity: 19.5+/−1.5 mS/cm). In this step the fibrinogen is eluted from the column. Most of the vWF still binds to the column.

The column may then be rinsed with a buffer with higher salt concentration (15 mM tri-sodium citrate dehydrate; 1.5 M sodium chloride, pH value: 6.5+/−0.1, conductivity: 113.5+/−5.0 mS/cm), eluting vWF from the column. The column may then be cleaned using 1 M sodium hydroxide.

In this method, while most of the albumin and IgG are already separated from the fibrinogen with the precipitation step, they also do not bind to the CEX material. More than 50% of the vWF present in the liquid phase containing fibrinogen can be removed by using this method.

For the production of a drug substance, the eluted fractions may be concentrated using ultrafiltration and the protein concentration adjusted to 33 g fibrinogen per liter by using citrate buffer. Further ingredients may be added to form the final drug substance, e.g., as described herein.

As described in steps a) and the following steps, the drug substance is then preferably filtrated (0.2 μm) into different containers, e.g., vials under the conditions described above, and dried, preferably, lyophilized, to a residual moisture content of 2-5% (w/w). Then, preferably, a final heat treatment in a steam autoclave (e.g., about 100° C., 30 min) is performed as a further virus inactivating step. The resulting product is surprisingly stable.

The method of the invention may be advantageously used for decreasing the number of SVPs, in particular, to the limits described herein. To this end, the gentle filling conditions are of particular relevance. The method of the invention may also be advantageously used for decreasing the number of viruses, e.g., non-enveloped viruses. To this end, the dry heat treatment at a residual moisture content of 2-5%, preferably, in the presence of polysorbate, e.g., polysorbate 80, is of particular relevance. In combination, the method of the invention leads to a particularly safe product for the patient.

The invention also provides a container comprising a fibrinogen drug product or batch of containers comprising fibrinogen drug product of the invention obtainable from the method of the invention, as described herein.

### Use, in Particular, Medical Use

In another embodiment, the invention provides a container comprising a fibrinogen drug product for use in treatment of fibrinogen-deficiency. For example, the fibrinogen drug product may be for use in treating a genetic fibrinogen-related disorder. Preferably, the drug product is for use in treating congenital fibrinogen-deficiency. The fibrinogen-deficiency may be e.g., congenital afibrinogenemia, congenital hypofibrinogenemia, fibrinogen storage disease, congenital dysfibrinogenemia, hereditary fibrinogen Aa-Chain amyloidosis, congenital hypodysfibrinogenemia or cryofibrinogenemia.

The fibrinogen drug product of the invention may also be for use in treating an acquired fibrinogen-related disorder such as acquired dysfibrinogenemia or acquired hypofibrinogenemia.

If the fibrinogen-deficiency is acquired, the drug product preferably is for use in treating blood loss or bleeding, optionally, during (severe) surgical procedures. The blood loss or bleeding may also be due to trauma.

The drug product of the invention may be administered instead of a plasma preparation such as fresh frozen plasma. It is advantageous to administer a fibrinogen drug product of the invention instead of such a plasma product, or instead of a fibrinogen product further comprising high amounts of other plasma proteins such as albumin, as, with the present product, the plasma proteins can be separately dosed according to the need of the patient. In particular, fibrinogen administration is indicated in case of bleeding, i.e., for treatment of bleeding, or to prevent bleeding in case there is a fibrinogen-deficiency. The drug product of the present invention is also particularly virus-safe.

In another embodiment, the invention provides a method for treating fibrinogen-deficiency, comprising administering a fibrinogen drug product of the invention to a patient in need thereof, e.g., a patient suffering from any of the above-mentioned conditions. The patient who is administered the drug product preferably is a human patient.

Typically, the fibrinogen drug product or batch of fibrinogen drug products of the invention is formulated for use in intravenous administration to a patient after resuspension. Because of the low number of SVPs, as defined herein, the fibrinogen drug product or batch of fibrinogen drug products of the invention advantageously is suitable for administration to a patient after resuspension and without previous filtration. This saves time, particularly, in emergency cases. However, the fibrinogen drug product may also be used after resuspension and filtration. A filter may be part of a kit of the invention comprising the container comprising the fibrinogen drug product of the invention and a filter, preferably, a transfer device or syringe-top filter. Filtration may help in resuspension of the product of the invention, in particular, to avoid or reduce formation of foam. Alternatively, to avoid formation of foam, the resuspension can be carried out by gentle shaking.

The fibrinogen drug product or batch of fibrinogen drug products of the invention may also be used as a fibrin glue, e.g., in bandages. Alternative uses are as part of a cell-culture medium or in organ printing, e.g., three-dimensional organ printing.

### EMBODIMENTS OF THE INVENTION

The invention provides, e.g., the following embodiments:

Embodiment 1 is a container comprising a fibrinogen drug product in dry form having a residual moisture content of 2-5% (w/w). In Embodiment 2, the container comprising the drug product of embodiment 1 has a residual moisture content of 2.5-3.5% (w/w), e.g., about 3% (w/w).

In Embodiment 3, the drug product in the container of any of embodiments 1 or 2 is suitable for reconstitution in an aqueous solution, preferably water for injection, and wherein a fibrinogen solution resulting from said reconstitution of 1 g of fibrinogen of said drug product comprises not more than 6000 SVPs having a size of 10-100 μm and not more than 600 SVPs having a size of 25-100 μm. In Embodiment 4, for the drug product in the container of any of embodiments 1-3, upon reconstitution of one container in a solvent, especially in water for injection, a fibrinogen solution comprising 6000 or less SVPs having a size of 10-100 μm and 600 or less SVPs having a size of 25-100 μm is obtained.

In Embodiment 5, the invention provides a container comprising a fibrinogen drug product in dry form, wherein the fibrinogen drug product is suitable for reconstitution in an aqueous solution, preferably water for injection, and wherein a fibrinogen solution resulting from said reconstitution of 1 g of fibrinogen of said drug product comprises not more than 6000 SVPs having a size of 10-100 μm and not more than 600 SVPs having a size of 25-100 μm. In Embodiment 6, for the drug product in the container of embodiment 5, upon reconstitution of one container in a solvent, especially in water for injection, a fibrinogen solution comprising 6000 or less SVPs having a size of 10-100 μm and 600 or SVPs particles having a size of 25-100 μm is obtained.

In Embodiment 7, the invention provides a batch of containers comprising fibrinogen drug product in dry form, wherein, for at least 10 containers from said batch, the fibrinogen drug product is suitable for reconstitution in an aqueous solution, preferably water for injection, and wherein a fibrinogen solution resulting from said reconstitution of 1 g of fibrinogen of said drug product comprises not more than 6000 SVPs having a size of 10-100 μm and not more than 600 SVPs having a size of 25-100 μm. In Embodiment 8, these conditions apply for at least 100 containers from the batch of Embodiment 7, optionally, at least 200 containers. In Embodiment 9, these conditions apply for at least 500 containers from the batch of embodiment 7, optionally, to substantially all containers of said batch. In Embodiment 10, these conditions apply for at least the 10 final % of containers from the batch of any of Embodiments 7-9, preferably, for at least the final 20% of containers from said batch, optionally, for substantially all containers of said batch.

In Embodiment 11, the container comprising fibrinogen drug product or batch of containers comprising fibrinogen drug product of any of embodiments 5-10 has a residual moisture content of 2-5% (w/w). In Embodiment 12, the container comprising fibrinogen drug product or batch of containers comprising fibrinogen drug product of any of embodiments 5-11 has a residual moisture content of 2.5-3.5% (w/w), e.g., about 3% (w/w).

In Embodiment 13, the fibrinogen drug product of any of embodiments 1-12 is packaged in a vial, i.e., the container is a vial. In Embodiment 14, the fibrinogen drug product in the container of any of embodiments 1-13 is lyophilized. In Embodiment 15, the container of any of embodiments 1-14 contains 1 to 3 g of fibrinogen, optionally, 1 g. In Embodiment 16, the reconstitution in any of embodiments 1-11 is at a concentration of 20 g fibrinogen/L.

In Embodiment 16, in the container comprising fibrinogen drug product or batch of containers comprising fibrinogen drug product of embodiments 1-15, the fibrinogen drug product is suitable for reconstitution in an aqueous solution, preferably water for injection, and wherein a fibrinogen solution resulting from said reconstitution of 1 g of fibrinogen of said drug product comprises not more than 3500 SVPs having a size of 10-100 μm and not more than 60 SVPs having a size of 25-100 μm. Consequently, in Embodiment 17, for the container comprising drug product of any of embodiments 1-16, upon reconstitution of one container in a solvent, especially in water for injection, a fibrinogen solution comprising 3500 or less SVPs having a size of 10-100 μm and 60 or less SVPs having a size of 25-100 μm is obtained.

In Embodiment 18, the fibrinogen drug product or batch of fibrinogen drug products in the container of any of embodiments 1-17 is suitable for reconstitution in an aqueous solution, preferably water for injection, and wherein a fibrinogen solution resulting from said reconstitution of 1 g of fibrinogen of said drug product comprises not more than 3500 SVPs having a size of 10-100 μm and not more than 35 SVPs having a size of 25-100 μm. Consequently, in Embodiment 19, for the drug product in the container of any of embodiments 1-18, upon reconstitution of one container in water for injection, a fibrinogen solution comprising 3500 or less SVPS having a size of 10-100 μm and 35 or less SVPs having a size of 25-100 μm is obtained.

In Embodiment 20, the fibrinogen drug product or batch of fibrinogen drug products in the container of embodiments 1-19 is suitable for reconstitution in an aqueous solution, preferably water for injection, and wherein a fibrinogen solution resulting from said reconstitution of 1 g of fibrinogen of said drug product comprises not more than 2500 SVPs having a size of 10-100 μm and not more than 30 SVPs having a size of 25-100 μm. Consequently, in Embodiment 21, for the drug product in the container of any of embodiments 1-20, upon reconstitution of one container in a solvent, especially in water for injection, a fibrinogen solution comprising 2500 or less SVPs having a size of 10-100 μm and 30 or less SVPs having a size of 25-100 μm is obtained.

In Embodiment 22, the fibrinogen drug product in the container or batch of containers comprising fibrinogen drug products of embodiments 1-21 comprises
a) polysorbate,
b) optionally, a bulking agent,
c) optionally, an amino acid and/or
d) optionally, a salt selected from the group comprising sodium chloride, sodium citrate and a combination thereof.

In Embodiment 23, the fibrinogen drug product comprised in the containers of embodiments 1-22 comprises polysorbate, optionally, polysorbate 80. In Embodiment 24, the fibrinogen drug product or batch of fibrinogen drug products comprised in the containers of embodiments 1-23 comprises a bulking agent, optionally, trehalose. In Embodiment 25, the fibrinogen drug product comprised in the containers of embodiments 1-24 comprises an amino acid, optionally, arginine.

In Embodiment 26, the fibrinogen drug product comprised in the containers of embodiments 1-25 comprises a salt selected from the group comprising sodium chloride, sodium citrate and a combination thereof, optionally, sodium chloride and sodium citrate.

In Embodiment 27, the fibrinogen drug product comprised in the containers of embodiments 1-26 comprises
a) polysorbate, preferably, polysorbate 80,
b) a bulking agent, preferably trehalose,
c) an amino acid, preferably arginine and
d) a salt selected from the group comprising sodium chloride, sodium citrate and a combination thereof.

In Embodiment 28, the fibrinogen drug product comprised in the containers of embodiments 1-27 comprises polysorbate 80, trehalose, arginine, sodium, chloride, citrate and residual moisture. In Embodiment 29, the fibrinogen drug product comprised in the containers of embodiments 1-28 further comprises less than 1 mM calcium. In Embodiment 30, the fibrinogen drug product comprised in the containers of embodiments 1-29 further comprises additional plasma proteins selected from the group comprising albumin, fibronectin, a2-macroglobulin, immunoglobulins such as IgG, IgA or IgM, Von Willebrand factor, fibrinopeptide A and D-Dimer. In Embodiment 31, the fibrinogen drug product comprised in the containers of embodiments 1-30 consists of the recited components, i.e., fibrinogen, a polysorbate such as polysorbate 80, a bulking agent such as trehalose, an amino acid such as arginine, a salt such as sodium chloride and/or sodium citrate, or plasma protein(s) selected from the group comprising albumin, fibronectin, a2-macroglobulin, immunoglobulins such as IgG, IgA or IgM, Von Willebrand factor, fibrinopeptide A and D-Dimer or consisting of said groups.

In Embodiment 32, the fibrinogen drug product comprised in the containers of embodiments 1-31 comprises upon reconstitution at 20 g/L fibrinogen, less than 0.5 g/L albumin, preferably, less than 0.15 g/L albumin. In Embodiment 33, the fibrinogen drug product comprised in the containers of embodiments 1-32 does not comprise saccharose and/or glutamate, preferably, neither saccharose nor glutamate. Preferably, none of the non-fibrinogen plasma proteins recited above is contained in a concentration higher than 0.5 g/L.

In Embodiment 34, the fibrinogen drug product comprised in the containers of embodiments 1-33 is stable at 2-25° C. for at least 6 months, preferably, for at least 1 year, optionally, for at least 5 years.

In Embodiment 35, the fibrinogen drug product comprised in the containers of embodiments 1-34 comprises less than 20% of aggregates, preferably, less than 10% of aggregates.

In Embodiment 36, the invention provides a method for producing a container comprising fibrinogen drug product of any of embodiments 1-35, comprising steps of
a) sterile filtering a bulk solution of fibrinogen drug substance,
b) receiving sterile filtered bulk solution in a receiving tank, optionally, comprising a stirrer having a stirring means,
c) optionally, stirring the bulk solution in the receiving tank when the stirring means is submerged in said bulk solution,
d) filling containers with a pre-determined amount of the solution, e.g., using a pump,
e) lyophilizing the solution in the containers to obtain a lyophilized drug product,
f) treating the lyophilized drug product with dry heat. If the solution is stirred in step c), the receiving tank comprises a stirrer having stirring means.

In Embodiment 37, the method of embodiment 36 further comprises
g) packaging the contains comprising the drug product.

In Embodiment 38, in the method of any of embodiments 36 or 37, the residual moisture content after lyophilisation is 2-5% (w/w), optionally, 2.5-3.5% (w/w), e.g., about 3% (w/w).

In Embodiment 39, the lyophilisation method of any of embodiments 36-38, comprises steps of
a) freezing at −29° C. or less, for at least 4 h, preferably, −50° C. or less,
b) primary stepwise drying at −10° C. or less and at least 40 μbar (4 Pa), wherein preferably, temperatures increase in each step, starting with less than −25° C. and at least 200 μbar (20 Pa),
c) secondary drying at 17 to 23° C. for at least 2 hours, wherein the residual moisture content after lyophilisation is 2-5% (w/w).

In Embodiment 40, the lyophilisation method of embodiment 39 comprises steps of
a) freezing at −52° C. or less, for 8 h or more,
b) primary stepwise drying, the first step being carried out at about −36° C. at about 280 μbar (28 Pa) for about 48 h, the second step being carried out at about −23° C. at about 70 μbar (7 Pa) for about 40 h, and the third step being carried out at about −10° C. at about 40 μbar (4 Pa) for about 78 h,
c) secondary drying at about 20° C. at about 10 μbar (1 Pa) for about 3-4 h.

In Embodiment 41, in the method of any of embodiments 36-40, the treatment with dry heat (step f) comprises heating the drug product to 100° C.+/−1.5° C. In Embodiment 42, in the method of any of embodiments 36-41, the treatment with dry heat is performed for 30+/−3 min. In Embodiment 43, in the method of any of embodiments 36-42, the treatment with dry heat is performed in a steam autoclave.

In embodiment 44, in the method of any of embodiments 36-43, the solution is not stirred in step d), but stirred in step c), e.g, for at most 1 hour, preferably, for at most 1 hour. In embodiment 45, in the method of any of embodiment 36-44, stirring in step c) is for at most 10 min, e.g., for at most 5 min. In embodiment 46, in the method of any of embodiments 36-45, the stirring is at most 150 rpm, wherein, preferably, the receiving tank has a volume of 50-150 L and cylindrical form, and the stirring means are stirring blades (e.g. in the form of blades or rods) affixed to a central drive shaft that rotates at most 150 rpm, e.g., at most 80 rpm. In embodiment 47, in the method of any of embodiments 36-46, the solution is not stirred when the stirring rods would break the surface of the solution and/or when foam would form.

In embodiment 48, in the method of any of embodiments 36-43, the solution is not stirred in steps c) or d).

In Embodiment 49, the invention provides a container comprising a fibrinogen drug product or a batch of containers comprising fibrinogen drug product of any of embodiments 1-35 obtainable from the method of any of claims 36-48.

In Embodiment 50, the invention provides a container comprising a fibrinogen drug product or a batch of containers comprising fibrinogen drug product of any of embodiments 1-35 or 49 for use in treatment of fibrinogen-deficiency. In Embodiment 51, the fibrinogen drug product in the container of embodiment 49 is for use in treating a genetic fibrinogen-related disorder. In Embodiment 52, the fibrinogen drug product in the container of any of embodiments 49-50 is for use in treating congenital fibrinogen-deficiency. In Embodiment 53, the fibrinogen drug product in the container of embodiments 49 is for use in treating an acquired fibrinogen-related disorder. In Embodiment 54, the fibrinogen drug product in the container of any of embodiments 50 or 53 is for use in treating blood loss/bleeding, optionally, during (severe) surgical procedures. In Embodiment 55, the fibrinogen drug product in the container of any of embodiments 50 or 53 is for use in treating blood loss/bleeding due to trauma.

In Embodiment 56, the fibrinogen drug product in the container of any of embodiments 50-55 is for use in intravenous administration to a patient after reconstitution. In Embodiment 57, the fibrinogen drug product in the container of any of embodiments 50-56 is for use in administration to a patient after reconstitution and without previous filtration.

In Embodiment 58, the method of any of embodiments 36-48 is used for decreasing the number of SVPs in the drug product, in particular, to the limits described herein. In Embodiment 59, the method of any of embodiments 36-48 is used for decreasing the number of viruses, e.g., nonenveloped viruses, in the drug product. In Embodiment 60, the method of any of embodiments 36-48 is used for decreasing the number of SVPs in the drug product, in particular, to the limits described herein, and for decreasing the number of viruses, e.g., non-enveloped viruses, in the drug product.

The invention is further illustrated, but not limited by the following examples and figures. All literature cited is herewith fully incorporated herein.

### DETAILED DESCRIPTION OF THE DRAWINGS

FIG. 1: Schematic representation of a preferred filling plant used in the method of the invention. (1): air flow; (2): pressure line; (3): feeding tank, e.g., 80 L; 4: final filter; 5: product flow; 6: receiving tank, e.g., 50 L; 7: stirrer, preferably, at up to 150 rpm; 8: stirring blades; 9: peristaltic pump, e.g., 290 rpm or lower; 10: container, e.g., vial; 11: freeze dryer

FIG. 2: Residual moisture has a significant impact on virus inactivation kinetics (I). Fibrinogen samples spiked with PPV were, in the presence and absence of polysorbate 80 (PS 80) (sample without polysorbate-H3, sample with 0.05% polysorbate in the reconstituted product-H5) lyophilised to a residual moisture content of <1% (w/w) and, partly, adjusted to other defined residual moistures as described in Example 4. A dry heat (DH) treatment was performed for all samples at 99° C. for 0 min, 30 min, 45 min or 60 min.

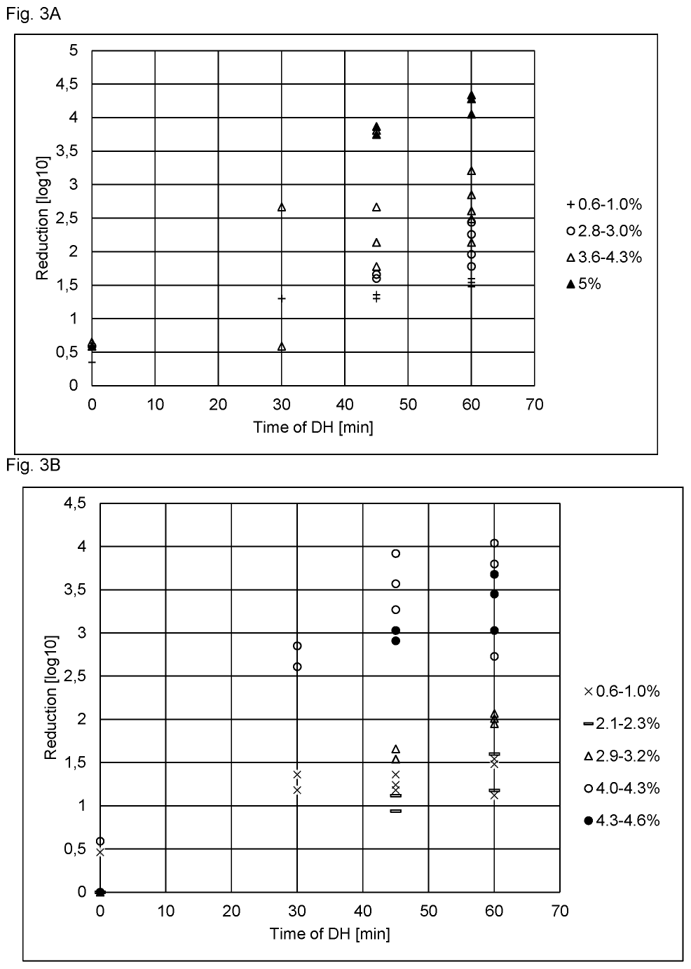

FIG. 3: Residual moisture has a significant impact on virus inactivation kinetics (II). Fibrinogen samples spiked with PPV were, in the presence and absence of polysorbate 80 (PS 80) (sample without polysorbate-H3, shown in FIG. 3A, sample with 0.05% polysorbate in the reconstituted product-H5, shown in FIG. 3B) lyophilised to a residual moisture content of <1% (w/w) and, partly, adjusted to other defined residual moistures as described in Example 4. A dry heat (DH) treatment was performed for all samples at 99° C. for 0 min, 30 min, 45 min or 60 min.

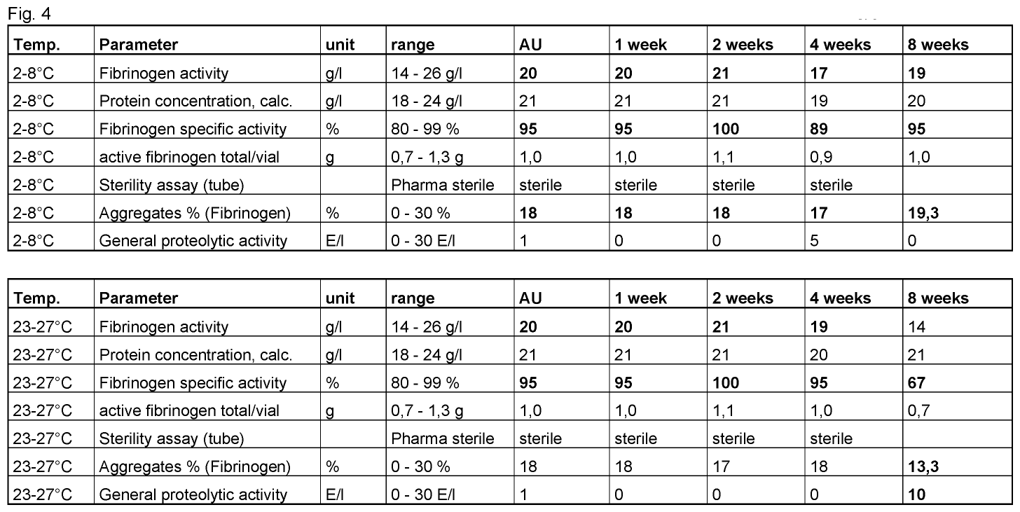

FIG. 4: Stability of reconstituted fibrinogen drug product of the invention at 2-8° C. and at 23-27° C. At 2-8° C., the fibrinogen drug product is stable for at least 8 weeks. At 23-27° C., the fibrinogen drug product is stable for at least 4 weeks (Study B).

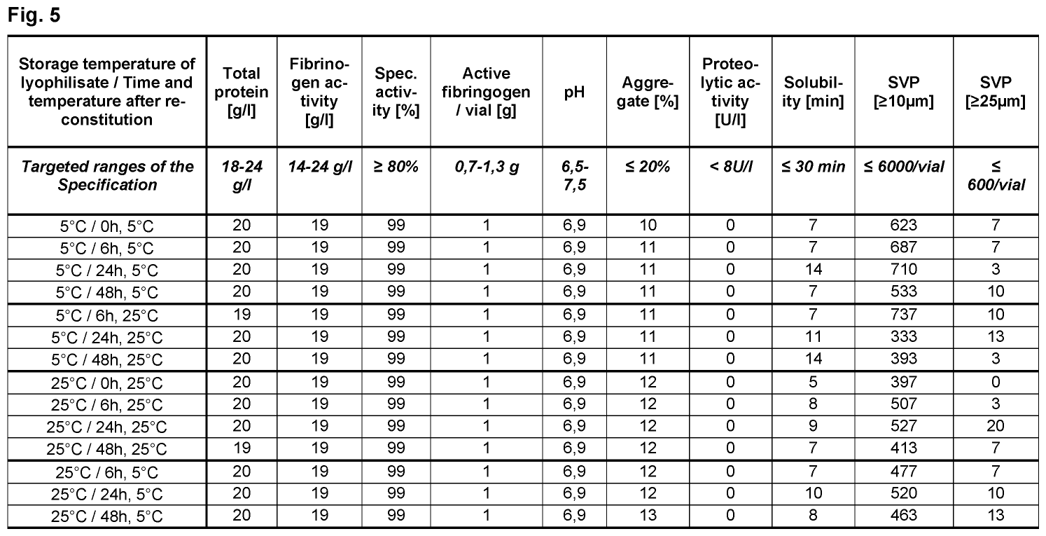

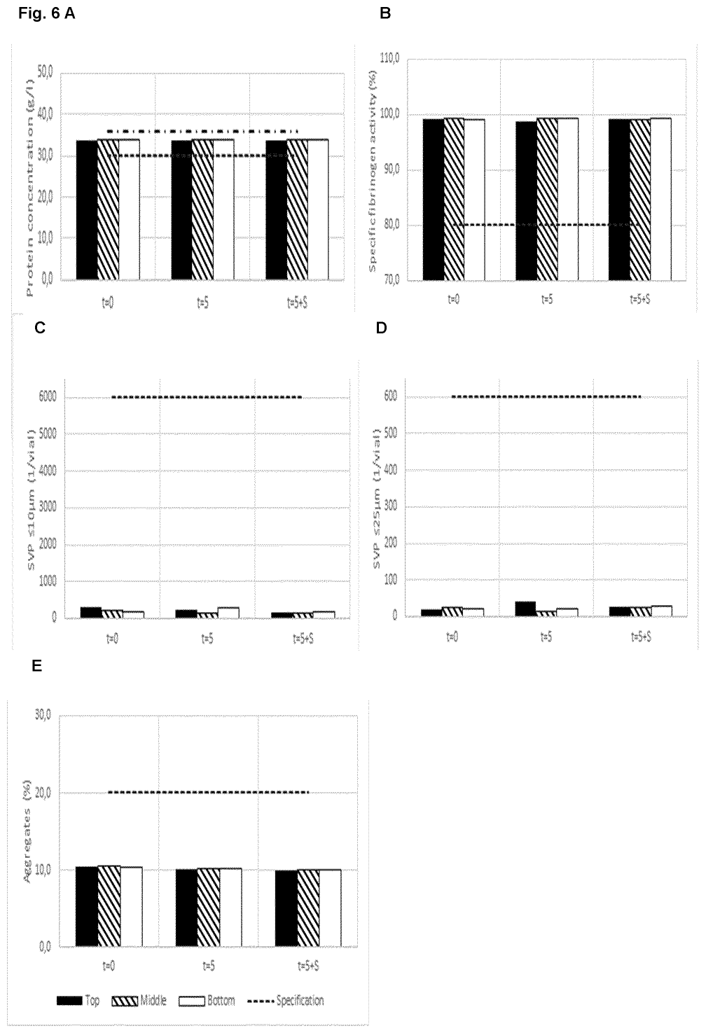

FIG. 5: Stability of the fibrinogen drug product of the invention after 6 years storage (Study C).

FIG. 6: Homogeneity of drug substance with and without stirring. The drug substance in the receiving tank was shortly stirred and samples taken from the surface, the middle and the bottom of the solution (t=0, left column group). After 5 h of incubation (t=5, middle column group), further samples were taken at the same locations. Then, the tank was again stirred, and more samples taken (t=5+S, right column group). There were no significant differences in protein concentration (A), specific fibrinogen activity (B), SVP≥10 μm (C), SVP≥25 μm (D) or aggregates (E), neither depending on the place from which the sample was taken, nor after incubation or stirring. In A and B, the dashed line is the specification lower limit and the irregular line in A is the specification upper limit. In C-E, the dashed line is the specification upper limit.

### EXAMPLES

### Methods

### Protein Determination

Protein determination was performed by the UV absorption method (Spektralphotometer Genesys™ 6, Spektralphotometer Genesys™ 10). Proteins in solution adsorb UV light at a wavelength of 280 nm due to the presence of aromatic amino acids, mainly tyrosine and tryptophan. This property is the basis of the protein determination at 280 nm. The accuracy of the UV spectroscopic determination of protein can be decreased by the scattering of light by the test specimen. For the compensation of this effect the absorption at 360 nm was subtracted from the absorption at 280 nm.

### Measurement of Fibrinogen (Fib:Ag)

The Fib:Ag concentration was determined by nephelometry at the BN Prospec (Siemens) Nephelometer. Fibrinogen forms a complex with a specific antibody. This complex causes a dispersion of irradiated light. The increased dispersion correlates to the fibrinogen concentration.

### Measurement of Activity of Fibrinogen by Clottable Protein

For the assay of fibrinogen activity (=clottable protein), the sample preparation was mixed with a suitable buffer solution containing sufficient thrombin and incubated at 37° C. The residual protein was determined in the supernatant by UV spectrometry at 280/360 nm and the result was subsequently subtracted from the total protein content (see above) to calculate the clottable protein.

### Determination of Specific Activity of Fibrinogen

The specific activity of fibrinogen was determined by clottable protein activity related to total protein as measured by UV absorption (280 nm).

Spiking with PPV

At 22±4° C., test material was spiked with the virus stock and aliquots of 34 ml were filled into containers, e.g., vials. Of the stock and the virus-spiked, test material, samples were taken and titrated. All vials were subjected to freeze drying. After freeze drying, the residual moisture in each container was determined by NIR (near infrared spectroscopy). For virus titration, the lyophilisates were suspended in 50 ml WFI (water for injection).

### Preparation of the Virus Inoculum

Virus stocks for PPV were prepared from virus-infected cells. For release of the virus stock for virus validation studies, the titer was verified by at least three independent titrations, using for each titration serial 3-fold dilutions and 8 replicates per dilution.

### Virus Titration

Samples were quantitatively analyzed for their virus content using a virus-specific cell-based infectivity assay (virus titration). After resuspension of the samples (where applicable), the samples were diluted and titrated immediately on the susceptible cell line. After a given incubation time, the virus-induced cytopathic effect was evaluated.

### Calculation Approach of Virus Titers

Virus titers were calculated, preferably, according to Spearman and Kaerber (Spearman C, Kaerber G. I In: Mayr A, Bachmann P A, Bibrack B, Wittmann G; eds. Virologische Arbeitsmethoden, Vol. I, p. 37-39 Fischer Verlag Stuttgart, 1974), or by applying the Poisson distribution (e.g., when no infectivity was detected).

### Calculation of Virus Reduction Factors

The virus reduction factor of each run was defined as the log 10 of the ratio of the virus load (total virus) in the virus-spiked test material and the virus load in the post-heat-treatment material. The virus load was calculated by multiplication of the concentration of virus titer with the volume.

### Measurement of Residual Moisture Content According to Karl Fischer and According to NIR Method (Near Infrared Spectroscopy)

These measurement methods were carried out as described in EP 0 844 005 A1.

### Measurement of Aggregate Content

In accordance with the Ph. Eur. 2.2.30 and USP 621, the aggregate content is measured by size exclusion chromatography with adjacent UV-detection. Therefore, the protein solution is applied onto the column resin, where the proteins interact with the porous resin in a size dependent manner. Small proteins or protein fragments show more intense interactions with the column resin and therefore have a longer resident time, while larger proteins or protein aggregations (dimers, trimers, etc.) have shorter resident times and are therefore eluted first. The protein fractions are detected by an UV-detector at the end of the column.

### Measurement of SVPs

In accordance with Ph. Eur. 2.9.19 and USP 788, the amount of SVPs is measured with light obscuration, e.g., by the particle counter Hiac Model 9703+ (Beckman, Krefeld, Germany) equipped with a Hiac Model HRLD-400 sensor 2 to 400 μm size range. For each measurement, protein solution is sucked through a capillary tube and passed by the sensor. The first measurement is used to flush the system and the results are discarded. The mean of the following four measurements is used to determine the number of SVPs in the solution. It is important to note that the protein solution is not filtered before the measurements.

### Example 1—The Production of Fibrinogen

### 1A) General Process Information

The following process is a preferred process for preparation of fibrinogen that may be used for preparation of the fibrinogen drug product used in the invention. Other processes can also be used, e.g., processes known in the art.

Fibrinogen concentrate drug substance (DS) may be manufactured, e.g., from a side fraction of a Factor VIII manufacturing process. The manufacturing process was performed under GMP conditions. The process comprised an anion-exchange chromatography step, wherein Factor VIII binds to the chromatography material and was further processed. The flow-through of the anion-exchange chromatography in the FVIII process was used for fibrinogen preparation and was collected and stabilized with tri-sodium-citrate. Subsequently, fibrinogen was precipitated by addition of glycine, NaCl and CaCl₂). The precipitate was separated by flow-through centrifugation. The harvested intermediate “glycine paste” could be stored at ≤−70° C. until further use.

For the manufacture of fibrinogen concentrate DS the frozen intermediate “glycine paste” was dissolved in sodium-citrate buffer (solution F01) and filtered. Subsequently, UV-C light irradiation was performed for virus inactivation. For further purification, the protein solution was chromatographically purified by cation exchange chromatography using POROS 50 HS. The collected column elution fraction was concentrated by ultra-filtration (UF). After addition of polysorbate 80 (PS 80) and trehalose, the pH and protein concentration were adjusted. The final bulk DS was further processed to the drug product (DP).

Fibrinogen concentrate DP was manufactured from the fibrinogen concentrate DS. The process was performed under GMP conditions. The formulated final bulk DS was filled into the final containers, wherein 32.0 ml protein solution were filled in 100 ml glass vials. Stoppers were loosely set onto the vials, which were then loaded on the pre-cooled shelves of a freeze-dryer and the freeze-drying process was started. After freeze-drying, the lyophilized product was heat treated in a steam autoclave for 30 min at 100° C. (product temperature) for inactivation of enveloped and especially non-enveloped viruses.

### 1B) Detailed Manufacturing Process of Fibrinogen Concentrate Drug Substance and Drug Product

The following listing gives an overview of the process steps for fibrinogen DS (Drug Substance) and DP (Drug Product) preparation in 1 g filling size.

### Step DS 1 Starting Material (Human Plasma)

### Step DS 2 Pooling and Thawing of Plasma

### Step DS 3 Separation of Cryo-Precipitate

Centrifugation
Temperature=2±2° C.
Storage of cryo-precipitate at ≤−25° C.

### Step DS 4 Pooling of Cryo-Precipitate

pH value=7.05±0.05
Temperature=22.5±2.5° C.
Stirring time≥30 minutes

### Step DS 5 Aluminium Hydroxide Treatment

Addition of 2% aluminium hydroxide suspension in 15-30 min
Stirring time=5 minutes
Temperature=22.5±2.5° C.
pH value=6.55±0.05
Temperature=15.0±1.0° C.

### Step DS 6 Polysorbate 80/TnBP Treatment (Virus Inactivation)

Protein≤10 g/l
Calcium chloride=0.001 M
pH value=7.1±0.1
Temperature=25.0±1.0° C.
Polysorbate 80=10±3 g/kg
Tri-n-butyl phosphate=3±0.9 g/kg
Stirring time=8-14 h
   Step DS 7 Anion exchange column chromatography
DEAE Toyopearl
Temperature=22±4° C.
Flow-through rate=192 l/h
   Step DS 8 Collection of flow-through
   Step DS 9 Glycine precipitation
Temperature=22±4° C.
0.0059 kg tri-sodium citrate per kg protein solution, stirring time≥5 min
0.091 kg glycine per kg protein solution, stirring time≥15 min
0.117 kg sodium chloride per kg protein solution and 0.000035 kg calcium chloride dihydrate per kg protein solution, stirring time≥5 min
Buffer A (equilibration and wash buffer of preceding anion exchange chromatography): 10 mM tri-sodium-citrate, 120 mM NaCl, 120 mM glycine, 1 mM CaCl₂), PH 7.0-7.1.
The resulting flow through was mixed with glycine and NaCl, precipitating fibrinogen (e.g., at room temperature, c (glycine): 1.2 M, c (NaCl): 2.0 M). A glycine paste was prepared by flow-through centrifugation.
The solid glycine paste was frozen, in particular, quickly frozen at ≤−50° C. (e.g., −50° C. to −70° C.), which reduces formation of aggregates.

### Step DS 10 Virus Inactivation by UV-C-Light Irradiation (Virus Inactivation)

Temperature=18±2° C.
UV-C dose=125-200 J/m², e.g., at 254 nm
Conductivity=9±1 mS/cm
The glycine paste was resuspended in sodium citrate buffer (Puffer F01), followed by filtration.
For irradiation an irradiation apparatus with a circumferential tube was used according to EP 0 840 624 B1. Irraditation at 254 nm with 125-200 J/m² was a particularly homogenous and gentle treatment for the product.

### Step DS 11 Cation Exchange Column Chromatography (CEX)

POROS 50 HS
Protein load=10-20 g/l resin per cycle
Conductivity=9±1 mS/cm
Temperature=22±4° C.
Linear flow rate=200±100 cm/h
The CEX reduced the concentration of vWF and further proteins and TnBP and polysorbate 80.
The column was equilibrated with the wash buffer F02 (15 mM citrate buffer (trisodium citrate), 65 mM NaCl, pH 6.5). Fibrinogen containing fractions were added and fibrinogen binds to the column. Elution was performed with arginine/citrate/NaCl with an elution buffer (F03: 7.5 mM citrate buffer (tri-sodium citrate), 150 mM NaCl, 75 mM arginine, pH 7.0).

### Step DS 12 Ultrafiltration

Protein conc.=55 g/l±10 g/l

### Step DS 13 Adjustment of the Protein Solution, 0.2 μm Filtration and Quality Control of the Drug Substance

Trehalose-dihydrate=0.0170±0.0017 per kg protein solution
Polysorbate 80=0.075±0.0075%
pH=7.0±0.5
Fibrinogen concentration=30-36 g/l
The protein content was adjusted to 33 g/l, and pH 7.0 was adjusted with citrate buffer. 0.017 kg trehalose/kg protein was added. 0.075% (w/v) polysorbate 80 (for stabilisation, for improving solubility, to increase the reduction of non-enveloped virus, e.g., parvovirus). Before lyphilisation, the concentration of polysorbate was adjusted to 0.075% polysorbate (w/v). After reconstitution this typically leads to a concentration of 0.05% polysorbate.
→Drug Substance (final Bulk)
   Step DP 1+2 Final Sterile Filtration and Filling into Final Containers/Sales Units
0.2 μm filtration
Filling of about 32.0 ml

### Step DP 3 Freeze-Drying

Freezing at ≤−29° C. for ≥4 h
Primary drying stepwise≤−10° C.
Secondary drying 20° C.±3° C. for ≤4 h
Total drying for about 150-200 h

### Step DP 4 Heat Treatment (Virus Inactivation)

product temperature: 100±1.5° C. for 30±3 min
Residual moisture 2.0-5.0%
Cooling to ≤80° C.

### Step DP 5+6 Quality Control of DP and Packaging

Storage at 5° C.±3° C.

The preferred process of production of the drug product is described in more detail in EP 19 214 919.3 (application number, not published yet), and in the critical steps of the invention, below.

### Example 2—The Filling Process

In a preferred process of the invention for final filling of fibrinogen concentrate drug substance (DS) a standardized/routine filling line for filling of pharmaceutical proteins was employed (Bausch & Ströbel, Ilshofen, Germany, KS 1025, model: AS 18.2). The setup is shown in FIG. 1.
a) For filling, the final DS was 0.2 μm-filtered using filter 4 (e.g., Filter type: Sartorius 5235307H9-SS, 0.2 m², cellulose acetate (CA) membrane) from the feeding tank 3 (typically, a stainless steel tank, e.g., with a nominal filling volume of 80 L) into a receiving tank 6 (typically, a stainless steel tank, e.g., with a nominal filling volume of 50 L) by pressure superposition in pressure line 2 and air flow 1 (e.g., at ≤600 mbar (600 hPa)) along the product flow 5. When the stirring bar 8 (or the stirring rods) of the stirrer 7 was covered with the fibrinogen solution, e.g., after filling of approximately 15 L of the receiving tank 6, the stirrer 7 was started at a low speed, especially with ≤150 rpm, preferably 80 rpm±20 rpm, ensuring a submerged stirring bar 8 of the stirrer. The protein solution surface was thereby kept above the stirring means 8, preventing foam formation. Then, e.g., after complete drug substance transfer into the receiving tank 6, filling was started, using a peristaltic pump 9 with a pump speed≤290 rpm, preferably ≤270 rpm. The glass vials 10 (nominal filling volume: 100 ml, diameter: 5.2 cm) were filled with 32 ml drug substance, each. The stirrer 7 was stopped when, or before the solution surface reached the rods/stirrer means 8.

A number of vials 10, especially 50 vials, were collected in a frame, especially a metal frame, which was transferred into the pre-chilled (≤−50° C.) freeze-dryer 11 without delay.

The resulting drug products could be reconstituted with a low number of SVPs formed, as described herein, which is due to the mild stirring conditions.

b) Alternatively, for filling, the final DS was 0.2 μm-filtered using filter 4 (e.g., Filter type: Sartorius 5235307H9-SS, 0.2 m², cellulose acetate (CA) membrane) from the feeding tank 3 (typically, a stainless steel tank, e.g., with a nominal filling volume of 80 L) into a receiving tank 6 (typically, a stainless steel tank, e.g., with a nominal filling volume of 50 L) by pressure superposition in pressure line 2 and air flow 1 (e.g., at ≤600 mbar (600 hPa)) along the product flow 5. After complete drug substance transfer into the receiving tank 6, the stirrer 7 was started at a low speed, especially with ≤150 rpm, preferably 80 rpm±20 rpm. The solution was stirred for 5 min, and then the stirrer was stopped. Filling was started, using a peristaltic pump 9 with a pump speed≤290 rpm, preferably ≤270 rpm. The glass vials 10 (nominal filling volume: 100 ml, diameter: 5.2 cm) were filled with 32 ml drug substance, each.

Remaining drug substance was filled into the receiving tank 6 before it was empty, so that with the remaining and added drug substance solution, the stirrer 7 could be used when submerged. The solution was stirred for 5 min, and then the stirrer was stopped and the filling process restarted. Alternatively, filling can be started while stirring, if it is ensured that the stirring means are always submerged when stirring.

A number of vials 10, especially 50 vials, were collected in a frame, especially a metal frame, which was transferred into the pre-chilled (≤−50° C.) freeze-dryer 11 without delay.

By taking samples from different stages of the filling process and comparing the samples, e.g., for concentration of fibrinogen, the inventors have surprisingly found that this mild and short stirring is sufficient for a homogenous product. Even without stirring, no heterogeneity was found.

The numbers of SVP were reproducibly even lower than in method a).

c) To compare homogeneity with and without stirring, the drug substance in the receiving tank 6 was shortly stirred and samples taken from the surface, the middle and the bottom of the solution. After 5 h of incubation (t=5), further samples were taken at the same locations. Then, the tank was again stirred, and more samples taken (t=5+S). There were no significant differences, neither depending on the place from which the sample was taken, nor after incubation or stirring (FIG. 6).

### Example 3—Lyophilisation

It is especially advantageous that the filled vials 10 were loaded into a pre-chilled freeze-dryer. This allowed for a quick freezing of the protein solution and resulted in a favorable, porous cake structure, which improved water sublimation. The lyophilisation was performed in a way that led to a residual moisture of 2.5-5% (w/w).

In a preferred process, after loading of the final vials, the drying program was started: The first step consisted of an 8 h freezing step at −52° C. followed by primary drying. Primary drying was subdivided in three steps: drying at 280 μbar (28 Pa) and −36° C. for 5-45 h (step 1), drying at 70 μbar (7 Pa) and −23° C. for 25-45 h (step 2) and drying at 40 μbar (4 Pa) and −10° C. for 45-78 h (step 3) (preferably, either step 1 for 45 h, step 2 for 45 h and step 3 for 45 h or step 1 for 5 h, step 2 for 25 h and step 3 for 78 hours). This procedure improved the solubility of the final product and reduced the amount of SVPs. In order to switch to secondary drying, a pressure rise test (0.044 μbar (0.44 Pa) within 3 min) needed to be passed. Secondary drying at 10 μbar (1 Pa) and 20° C. for 3 h decreased the variation of the residual moisture and led to a more homogenous lyophilisate behavior after heat treatment (30 min at 100° C.). In addition, secondary drying at 20° C. lowered the amount of SVPs in the final product.

The residual moisture was tested for each batch using the NIR or Karl-Fischer method. NIR results can be correlated to the Karl-Fischer method using a table calibrated for the samples of the invention. For analysis, the water was extracted from the lyophilisate and chemically quantified with a following reaction.

As shown in Table 1, the residual moisture content in products prepared with the method of the invention typically was between 2.5-3.5% (w/w), as detected with the Karl-Fischer method. Comparison with commercially available products showed significantly lower residual moisture content, which are lower than 1% (w/w).

TABLE 1
Residual moisture in comparative samples

| | | Residual moisture |
|---|---|---|
| | | % (w/w) according |
| | | to Karl Fischer |
| Sample | Source of sample | method |
| Fibrinogen_1 g_Fib_1169 | Fibryga 1 g from | 0.65 |
| sample 1 | Octapharma | |
| | Ch.-B.: K8/3°3491 | |
| Fibrinogen_1 g_Fib_1169 | Haemocomplettan P | 0.35 |
| sample 2 | 1 g from | |
| | CSL Behring | |
| | Ch.-B.: P100015559 | |
| Fibrinogen_1.5 g_Fib_1169 | FibClot 1.5 g from | 0.08 |
| sample 3 | LFB | |
| | Ch.-B.: 16L08426 | |

### Example 4—Comparison of Virus Inactivation Depending on the Residual Moisture Content

Enveloped viruses were reliably eliminated by the solvent/detergent step carried out during preparation of the drug substance as described above. Non-enveloped viruses should be further eliminated. Optimal conditions were analysed in the following experiment.

For the experiments with non-enveloped viruses, first, fibrinogen samples spiked with PPV were, in the presence and absence of polysorbate 80 (PS 80) (sample without polysorbateH3, sample with 0.05% polysorbate in the reconstituted product-H5) lyophilised to a residual moisture content of <1% (w/w). The moisture of samples after freeze-drying was measured by NIR (near-infrared spectroscopy).

Some samples were then again opened to add a defined amount of water for injection (WFI) into the vial, not touching the lyophilisate. The vial was closed again with a stopper and was incubated until the applied WFI was evaporated. In order to get a comparable pressure in all vials, the vial was opened and set into the chamber of the freeze-dryer. The vial was then closed under vacuum. The moisture content was measured again by NIR or in parallel control samples after extraction with an organic solvent according to the Karl Fischer method, as described herein.

A dry heat (DH) treatment was performed for all samples at 99° C. for 0 min, 30 min, 45 min or 60 min. 0 min represents a lyophilised sample without dry heat treatment. The reduction of the virus titer was calculated in logarithmical (log₁₀) terms, wherein, as a starting value for the calculation of reduction, material before lyophilisation was used. There was no significant virus reduction at 0 min. With an increase in the time of heating (30 min, 45 min, 60 min), an increased virus inactivation was seen at a residual moisture of 4% (between log 10 about 2,5-4, with one outlier value after 30 min heat treatment with a virus reduction factor just below 2 log₁₀ 2). Virus inactivation for the samples having a residual moisture lower than 1% was in the range of 1-1,5 log₁₀ for all time points. At this low moisture, no effect of polysorbate 80 could be seen. At higher residual moisture, there was a trend towards a positive effect of polysorbate 80. At all points of time, the highest reduction was obtained with a sample having about 4% residual moisture and comprising polysorbate (FIG. 2).

In a further set of experiment, the spiking experiments were repeated with another sample of fibrinogen, wherein other residual moisture contents between 0 and 5%, measured and adapted after lyophilisation, as described above, were used. Samples without (H3) and with polysorbate 80 (H5-0.05% in the reconstituted product) were tested.

The relation between residual moisture and virus inactivation, shown for PPV, could be confirmed both for the material without and for the material with PS 80.

In FIG. 3A and FIG. 3B, the samples are grouped according to their residual moisture, and virus reduction is shown for dry heat (DH) treatment at 99° C. for different times. In the samples with 45 or 60 min DH, a significantly higher virus inactivation could be shown for the more moist samples. In the presence of PS 80 (FIG. 3B), or more than 3.2 to 4.3%, or preferably, at least 4% residual moisture appeared to be optimal for a significant efficacy of virus inactivation, while without polysorbate (FIG. 3A), 5% residual moisture were optimal.

### Example 5—Formulation of the Drug Product

One container, e.g., one vial of fibrinogen drug product of the invention, upon reconstitution in 50 mL water for injection at 20 g/L, which is suitable for intravenous administration may, e.g., have the following characteristics:

| pH | 7.0 | |
|---|---|---|
| Osmolarity | ≥240 | mosmol/kg |
| Protein | 20 | g/L |
| Spec. activity | ≥80%, e.g., 98% ± 0.8% (n = 7) | |
| Na | 95-135 mmol/L, e.g., about 115 mmol/L | |
| Ca | <1 | mmol/L |

| | |
|---|---|
| Cl | 100-160 mmol/L, e.g., about 130 mmol/L |
| Citrate | 3-7 mmol/L, e.g., about 5 mmol/L |
| Arginine | 25-55 mmol/L, e.g., about 40 mmol/L |
| Trehalose | 22-38 mmol/L, e.g., about 30 mmol/L |
| Polysorbate 80 | 0.03-0.07%, e.g., about 0.05% |

content of aggregates≤20%, e.g., 11±1% (n=11) (determined by HP-SEC analysis)

TABLE 2
Overview of excipients in a preferred fibrinogen
drug product of the invention

| | Amount/1 mL | | |
|---|---|---|---|
| | reconstituted | Reference | |
| Component | solution | Standard | Function |
| Citrate | 0.96 mg | Ph. Eur. | Stabilizer |
| Sodium | 2.64 mg | Ph. Eur. | Electrolyte |
| Chloride | 4.61 mg | Ph. Eur. | Electrolyte |
| Arginine | 6.97 mg | Ph. Eur. | Stabilizer |
| Trehalose | 10.27 mg | Ph. Eur. | Stabilizer |
| Polysorbate 80 | 0.5 mg (0.05%) | Ph. Eur. | Stabilizer |
| Water for Injection | Ad 1 mL | Ph. Eur. | Solvent |

TABLE 3
Additional plasma proteins that may be comprised
in the drug product of the invention

| | Source of | Mean content | |
|---|---|---|---|
| Parameter | impurity | (3 batches) | |
| Albumin | plasma | 0.09 | g/l |
| Fibronectin | plasma | 0.27 | g/l |
| α2-macroglobulin | plasma | 0.14 | g/l |
| IgG | plasma | 0.06 | g/l |
| IgA | plasma | 0.15 | g/l |
| IgM | plasma | 0.20 | g/l |
| Von Willebrand factor | plasma | 4.1 | U/ml |
| Fibrinopeptide A | plasma | 9.2 | μg/l |
| D-Dimer | plasma | 0.19 | mg/1 |

### Example 6—Measurement of SVPs

According to Ph. Eur. 2.9.19 and USP 788, the amount of SVPs was measured as described above.

For the final fibrinogen concentrate drug product (fibrinogen drug product, DP), the numbers of particles≥10 μm and ≥25 μm were determined. The freeze-dried DP was reconstituted with 50 mL water for injection (WFI). The reconstituted solution was transferred to the particle counter without any further filtration. Every sample was measured three times. The mean value of the total particle amount per vial was reported. As the particles≥25 μm are also included in the group of particles≥10 μm, the number of particles≥10 μm was always higher than the number of particles≥25 μm.

The results of the last five batches produced by the method of the invention showed that the number of SVPs in the final product were always within the specification (Table 4). Accordingly, no additional filtration is needed before administration.

TABLE 4
Number of SVPs ≥10 μm per vial and ≥25 μm per
vial determined in the release measurement of the final
DP (five exemplary batches) generated by the method of Example 2a

| | B524016 | B524026 | B524017 | B524028 | B524019 |
|---|---|---|---|---|---|
| SVPs | 1666 | 2274 | 1642 | 1870 | 1111 |
| ≥10 μm | | | | | |
| per vial | | | | | |
| SVPs | 23 | 26 | 19 | 30 | 4 |
| ≥25 μm | | | | | |
| per vial | | | | | |

For comparison purposes, the following Table 5 shows the SVP count analyzed in some competitor products.

TABLE 5
SVP count (particles per container), comparative products

| | SVP ≥10 μm | SVP ≥25 μm |
|---|---|---|
| Fib Clot ® (LFB) | 11633 | 500 |
| Fibryga ® (Octapharma) | 13626 | 463 |
| Fibryga ® (Octapharma) | 16917 | 167 |
| Fibryga ® (Octapharma) | 16800 | 143 |
| Haemocomplettan ® (CSL Behring) | 17640 | 1053 |
| Haemocomplettan ® (CSL Behring) | 10947 | 264 |

The low amounts of SVPs in the product of the invention preferably result from the final formulation, in particular, the presence of polysorbate, the gentle filling process and the freeze-drying process, which have been precisely adapted to fibrinogen.

### Example 7—Stability of the Drug Product

### 7.1 Study A-Long-Term Stability of the Lyophilized Drug Product

To investigate the long-term stability of the fibrinogen drug product four batches of the fibrinogen drug product have been stored for stability testing. One batch was stored for 36 months and three batches were stored for 60 months, each at 5° C. (±3° C.) and at 25° C. (±2° C.). Based on this data a shelf life of at least 60 months was shown at 5° C. (±3° C.) and at 25° C. (±2° C.) for the fibrinogen drug product.

For stability testing the batches were tested for activity after reconstitution at predefined time points for each storage condition (5° C., 25° C., 55-65% relative humidity (RH)), i.e. the fibrinogen activity was tested in the reconstituted aqueous solution right after reconstitution and after 6 h and 24 h incubation at room temperature (approx. 25° C.). This investigation simulated the use of fibrinogen concentrate in clinical routine.

Storage at 5° C. (+3° C.): All results for the stability indicating parameters remained basically unchanged after 60 months (batch B524071, B524084 and B524032) and 36 months (batch B524031), respectively, i.e. the activity of the reconstituted fibrinogen, the pH value, the osmolality, the color and the opalescence remained unchanged. The solubility time of the lyophilizate varied between 5 min and 30 min which was acceptable. No increase of aggregates was detectable. For all time points, the stability of the reconstituted solution has been shown for up to 24 hours at room temperature. The content of excipients like citrate, trehalose and arginine remained almost unchanged. All tested solutions were sterile and free of pyrogens. The content of residual water measured within the batches was from 2.1 to 3.0% after 36 months respectively 60 months. The values were approximately 0.2% to 0.3% percentage points lower than before.

Storage at 25° C. (+2° C.): All results for the stability indicating parameters remained basically unchanged after 60 months (batches B524071, B524084 and B524032) and 36 months (batch B524031), respectively, i.e. the activity of fibrinogen, the pH value, osmolality, color and opalescence remained basically unchanged. The solubility time varied between 5 min and 30 min, a result which was acceptable.

All samples tested from the stability study were sterile and free of pyrogens for all storage conditions. There was no enrichment of aluminum during 60 months of storage at 5° C. (+3° C.) and 25° C. (+2° C.).

In summary, it was shown that the fibrinogen activity remained stable at least for the first 24 h after reconstitution by comparing the results obtained right after reconstitution as well as 6 h and 24 h later for each predefined time point over a shelf-life period of 60 months at a storage temperature of 5° C. and 25° C.

### 7.2 Study B—Stability of the Reconstituted Drug Product

For batch no. B524071, a stability study was carried out, wherein, after reconstitution, the drug product was stored at 2-8° C. or 23-27° C. for up to 8 weeks (FIG. 4). The determined residual moisture of this batch was 2.4%.

With a storage at 2-8° C., for up to 8 weeks, there was no increase of proteolytic activity or the content of aggregates, and no significant decrease of specific activity. With storage at 23-27° C., the samples were also stable for at least 4 weeks. Only after 8 weeks, the specific activity slowly decreased and the proteolytic activity increased.

### 7.3 Study C—Stability of the Reconstituted Drug Product (Stored for 6 Years)

A third stability study was carried out, wherein a six years old fibrinogen drug product (Batch no. B524032, stored at 5 or 25° C., respectively, for over six years) was tested for activity after reconstitution in water for injection at 5° C.±3° C. and 25° C.±2° C. (60% RH±5% RH) after defined time periods, i.e. 0 h, 6 h, 24 h and 48 h. The results are shown in FIG. 5.

Microbial testing after sampling was performed after defined time points in order to reveal microbial contamination during reconstitution. No sample showed microbial contamination. The pH of all samples was 6.9. The opalescence and the color was the same in all samples.

Concluding, even after six years (72 months) of storage of the dry drug product, the reconstituted drug product showed no loss of fibrinogen activity. Moreover, the reconstituted drug product was still stable even after 48 hours at 5° C. and at 25° C. All tested parameters have been shown to be within the targeted ranges of the specification, both direct after reconstitution and at all tested time points thereafter. Slight fluctuations were observed only for parameters “total protein”, “aggregates” and “sub-visible particles”. This high stability is surprising, even more so in the absence of stabilizing factors, e.g. substantial amounts of albumin, as described herein.

### Example 8—Reconstitution Characteristics

The comparatively high residual moisture was shown not to have a significant detrimental effect on solubility of the drug product. In comparison with other products, the product of the invention has a similar solubility.

The process of reconstitution or dissolution is typically finished within 3-8 min for the product of the invention, dissolved in water for injection. FibCLOT®, from LFB SA (Courtaboeuf Cedex, France) is dissolved within about 4-5 min, wherein a strong formation of foam is observed. Fibryga® von Octapharma (Langenfeld, Switzerland) is dissolved after 8 min. Haemocomplettan® from CSL Behring (Marburg, Germany) is dissolved after about 10 min. The method of solution itself (direct addition of water into the vial or Mix2Vial® (addition of water through a filter and filtration of the dissolved product), shaking by hand or use of a shaker) do not appear to have a significant influence on the result, wherein, generally, the formation of foam is lower with the Mix2Vial® method.

In summary, the lyophilisation and dry heat treatment according to the method of the present invention in combination with the filling process and the formulation leads to a virus-safe, active and highly soluble product.

1. A container comprising a fibrinogen drug product in dry having a residual moisture content of 2.5-3.5% (w/w).

2. The container of claim 1, wherein the fibrinogen drug product is suitable for reconstitution in an aqueous solution, and wherein a fibrinogen solution resulting from said reconstitution of 1 g of fibrinogen of said drug product comprises not more than 6000 SVPs having a size of 10-100 μm and not more than 600 SVPs having a size of 25-100 μm.

3. A container comprising a fibrinogen drug product in dry form, wherein the fibrinogen drug product is suitable for reconstitution in an aqueous solution, and wherein a fibrinogen solution resulting from said reconstitution of 1 g of fibrinogen of said drug product comprises not more than 6000 SVPs having a size of 10-100 μm and not more than 600 SVPs having a size of 25-100 μm.

4. A batch of containers comprising a fibrinogen drug product in dry, preferably, lyophilized form, wherein, for at least 10 containers from said batch, the fibrinogen drug product is suitable for reconstitution in an aqueous solution and wherein a fibrinogen solution resulting from said reconstitution of 1 g of fibrinogen of said drug product comprises not more than 6000 SVPs having a size of 10-100 μm and not more than 600 SVPs having a size of 25-100 μm.

5. The container comprising the fibrinogen drug product claim 3, wherein said fibrinogen drug product has a residual moisture content of 2.5-3.5% (w/w).

6. The container of claim 1, wherein the fibrinogen drug product of comprises
a) a polysorbate,
b) a bulking agent,
c) an amino acid
d) a salt.

7. The container comprising the fibrinogen drug product of claim 2, wherein the reconstitution is at a concentration of 20 g/L.

8. The container comprising the fibrinogen drug product claim 2, comprising, upon reconstitution at 20 g/L fibrinogen, less than 0.5 g/L albumin and no saccharose or glutamate.

9. The container comprising the fibrinogen drug product of claim 1, wherein the fibrinogen drug product is suitable for reconstitution in an aqueous solution, and wherein a fibrinogen solution resulting from said reconstitution of 1 g of fibrinogen of said drug product comprises not more than 3500 SVPs having a size of 10-100 μm and not more than 35 SVPs having a size of 25-100 μm.

10. The container comprising the fibrinogen drug product claim 1 that is stable at 2-25° C. for at least 6 months.

11. The container comprising the fibrinogen drug product claim 1 comprising less than 20% of aggregates.

12. A method for producing a container comprising the fibrinogen drug product of claim 1, comprising steps of
a) sterile filtering a bulk solution of fibrinogen drug substance,
b) receiving sterile filtered bulk solution in a receiving tank, optionally, comprising a stirrer having a stirring means,
c) optionally, stirring the bulk solution in the receiving tank when the stirring means is submerged in said bulk solution,
d) filling a container with a pre-determined amount of the solution,
e) lyophilizing the solution in the container to obtain a lyophilized drug product,
f) treating the lyophilized drug product with dry heat, and
g) optionally, packaging the container comprising the drug product.

13. The method of claim 12, wherein the receiving tank has a volume of 50-150 L and cylindrical form, and the stirring means are stirring blades (or rods) affixed to a central drive shaft that rotates at most 150 rpm, wherein the time of stirring is at most 1 hour,
wherein stirring in step c) is for at most 10 min and wherein the bulk solution is not stirred during step d).

14. The method of claim 12, wherein the lyophilisation comprises steps of
a) freezing at −29° C. or less, for at least 4 h,
b) primary stepwise drying at −10° C. or less and at least 40 μbar (4 Pa), wherein temperatures increase in each step, starting with less than −25° C. and at least 200 μbar (20 Pa),
c) secondary drying at 17 to 23° C. for at least 2 hours,
wherein the residual moisture content after lyophilisation is 2-5% (w/w), and
wherein the lyophilization optionally comprises
a) freezing at −52° C. or less, for 8 h or more,
b) primary stepwise drying, the first step being carried out at about −36° C. at about 280 μbar (28 Pa) for about 48 h, the second step being carried out at about −23° C. at about 70 μbar (7 Pa) for about 40 h, and the third step being carried out at about −10° C. at about 40 μbar (4 Pa) for about 78 h, and
c) secondary drying at about 20° C. at about 10 μbar (1 Pa) for about 3-4 h.

15. The method of claim 12, wherein the treatment with dry heat comprises heating the drug product to 100° C.+/−1.5° C. for 30+/−3 min.

16. A container comprising a fibrinogen drug product produced by the method of claim 12.

17. A method for treating fibrinogen-deficiency in a subject, comprising
(a) providing the container comprising the fibrinogen drug product of claim 1;
(b) reconstituting the fibrinogen drug product in an aqueous solution; and
(c) administering to the subject an effective amount of the fibrinogen drug product.

18. The container of claim 1 comprising the fibrinogen drug product in lyophilized form.

19. The container of claim 1, wherein the drug product has a residual moisture content of 2.5-3.5% (w/w).

20. The container of claim 3 comprising the fibrinogen drug product in lyophilized form.

21. The container of claim 3, wherein said fibrinogen drug products has a residual moisture content of 2.5-3.5% (w/w).

22. The batch of containers of claim 4, wherein said fibrinogen drug products have a residual moisture content of 2-5% (w/w).

23. The container of claim 6, wherein the amino acid is arginine.

24. The container of claim 6, wherein the drug product comprises polysorbate 80, trehalose, arginine, sodium, chloride, citrate and residual moisture.

25. The container of claim 9, wherein the fibrinogen drug product is suitable for reconstitution in an aqueous solution, and wherein a fibrinogen solution resulting from said reconstitution of 1 g of fibrinogen of said drug product comprises not more than 2500 or less SVPs having a size of 10-100 μm and not more than 30 or less SVPs having a size of 25-100 μm.

26. The method of claim 13, wherein stirring in step c) is for at most 10 min and wherein the bulk solution is not stirred during step d).

27. The method of claim 14, wherein the lyophilization comprises
a) freezing at −52° C. or less, for 8 h or more,
b) primary stepwise drying, the first step being carried out at about −36° C. at about 280 μbar (28 Pa) for about 48 h, the second step being carried out at about −23° C. at about 70 μbar (7 Pa) for about 40 h, and the third step being carried out at about −10° C. at about 40 μbar (4 Pa) for about 78 h, and
c) secondary drying at about 20° C. at about 10 μbar (1 Pa) for about 3-4 h.
